SEQUENCE LISTING (31 sequences)

SEQ ID NO: 1 - protein, 2026 aa, Gallibacterium anatis, strain 12656-12
  MISC_FEATURE (1)..(2026): Full length GtxA polypeptide
MLSLKEKVTGIDFDAIKDKVVSLKNTVSNIDFNLVKEDISSLKSNALSIAASDFKNKPVLFKDSLDLLTDATNTLRKITNQMSSISEISNKSLDLLDSLFEAAKDIVNIAYSKGGVEITKSATELAAKAALIVDKSIILANKDNTISEAVYHSINNSLQNIQKTAINIATHSHNEDKAEIAKASFELLSQVSDVISNALKNSGDIGIESQLLADINQFSHSILNTAKTVTDIATMDMNDKTSIAKNSISLIANVNDVISDILVMTDKDTELLNAIHNVTAKNLQNIEESAVNLANADVLSQEGKVSIAINSLTLISQTNKIVAQVLNEANLSTDKTQFVGELTDVLLNTAKSITLLATGNNATTAGKEQLAVASTNLIGNVNDLIQSITSFKGKEDIGNALHSAVDGQLSQIKQLAVALSNSNLDSSQGKTAIAITSFGLIAQANNIINKFLDNMSLSTNVSKSVHSLTNSALDAAKILTNVVQVDANNNQGKVVIANSSLELSKTASDIVSTVLKSTSISTQHIDIIHNAVNKTLTEMKDSAVAIALASSENNSAEIATHSLSLLSDASNMLKDIMQGMSPNNVIAPKTLELFNSLFATAQNIVQLADAKSSENIAKASVDLVQSATIILNNVLTLANVDSSLSKAFHQSFDASVSQIKEVAAQLATASSASNKAEIAKLSFDFISQVSDLATNTLTTAKTGLDSTLLNNVNGLSHSVLNAAKSVTDIIVSDNPANTASLSVSLVNNANEIVSNILTLSGKQNTLSTAVHDVTAKHLAPIEKIAINLANADNSSSDGKVAIALNSLTLIAQSNHLIEEVLKEAKLDNAKSAFAHNLTDLVLDTAKTITALASADTSKVDGKQQIASASTHLVGQINEIVKSITTITNSETKVGNAAYQALKTHLEQVETIAVKLAAANASTAEGRTEIAIESFNLIAKTNGIMTDFLNQIGIKEELTKPIQGLSNSILDTAKTLTYVVQIDPTTDKGKLSIADSSFELAKSANQIVSYIMDLSGSSSELSHNIANTAHQILSISQDRLLSIGNNISALANADKLTKEGVKIIVDSSFAITSDVNGFITDVVKTVGKDGNPKVGSALSLSNSIIDMGHSIANLIQSDVNTSSGKAAIAEGSIKLIGNINGLVSDVLSLSNASTAVSEAISSSAGGILTNLSSLIGSSIKLHNWSNMTQADQIAVGFDIGLKAVSTIATGVGTTAQSIAKIIGTTTMLPQIGAAVSGIALAASPLEIKGLVDEHKYVKQIDSLASETKTYGYQGDELLASLLNEKFALNTAYTATDIALNLATTAISVAATASVIGAPIAAIAGVVRGAIGGIMSAIKQPALEHIAKRYVDQIEKYGDIQKYFDQNTEATLNKFYASQEVIQSFKQLQKLYNVDNIITLDGVASSDSAIELAAITKLVEQMNKANNYAQLIRNGEIDKALSAQYLSMDAKTGVLEITAPGNSLIKFNSPLFAPGVEEARRKAVGKNNFYTDLIINGPNEHTINDGAGNNIFISNDKYASVLYDENGKLLKHINLNINAGDGNDTYIADNGHSLFNGGNGTDSVSYNNEHIHGIVVHGRDAGTYSVTKHIADAEVTVENIKVKNHQYGKRQERVEYRELHIETKSYDASDMLYNVEVISASDYDDVMYGSKGNDYFLAQNGNDLVYGKEGDDIIFGGAGDDKLYGETGNDTLNGGLGKDLIYGGEGNDTIIQDDALSSDTIFGDEGIDTLDLRHLVINDEGLGVVADLQSEKLYKGTIFDHIYDIENIIGTSGNDNLIGNHKDNILIGNDGDDILEGYSGNDVLAGGSGINKLYGGQGADIYLLSTNATNYIFDLTKNNLAKLENSEDLNLQFTKDSDDNVTLSFNKDGNTIGKTIIEKSSQFGTFSIGDGYYLDLNDGKFKYILSGESSNADLAQNTLHFNKGEELQVHAAAKDNQIILDHEHQHYINIYSNTQTNIKGFEVGKDKLQLSLLSNNLSSDTKLKFSGYDIEGGDVNITSGNTYITLSGAGHTDYASKTFNELVTMYLV

SEQ ID NO: 2 - protein, 1077 aa, Gallibacterium anatis, strain 12656-12
  MISC_FEATURE (1)..(1077): C-terminal part of GtxA polypeptide (amino acids 950-2026 of full length GtxA)
QIGIKEELTKPIQGLSNSILDTAKTLTYVVQIDPTTDKGKLSIADSSFELAKSANQIVSYIMDLSGSSSELSHNIANTAHQILSISQDRLLSIGNNISALANADKLTKEGVKIIVDSSFAITSDVNGFITDVVKTVGKDGNPKVGSALSLSNSIIDMGHSIANLIQSDVNTSSGKAAIAEGSIKLIGNINGLVSDVLSLSNASTAVSEAISSSAGGILTNLSSLIGSSIKLHNWSNMTQADQIAVGFDIGLKAVSTIATGVGTTAQSIAKIIGTTTMLPQIGAAVSGIALAASPLEIKGLVDEHKYVKQIDSLASETKTYGYQGDELLASLLNEKFALNTAYTATDIALNLATTAISVAATASVIGAPIAAIAGVVRGAIGGIMSAIKQPALEHIAKRYVDQIEKYGDIQKYFDQNTEATLNKFYASQEVIQSFKQLQKLYNVDNIITLDGVASSDSAIELAAITKLVEQMNKANNYAQLIRNGEIDKALSAQYLSMDAKTGVLEITAPGNSLIKFNSPLFAPGVEEARRKAVGKNNFYTDLIINGPNEHTINDGAGNNIFISNDKYASVLYDENGKLLKHINLNINAGDGNDTYIADNGHSLFNGGNGTDSVSYNNEHIHGIVVHGRDAGTYSVTKHIADAEVTVENIKVKNHQYGKRQERVEYRELHIETKSYDASDMLYNVEVISASDYDDVMYGSKGNDYFLAQNGNDLVYGKEGDDIIFGGAGDDKLYGETGNDTLNGGLGKDLIYGGEGNDTIIQDDALSSDTIFGDEGIDTLDLRHLVINDEGLGVVADLQSEKLYKGTIFDHIYDIENIIGTSGNDNLIGNHKDNILIGNDGDDILEGYSGNDVLAGGSGINKLYGGQGADIYLLSTNATNYIFDLTKNNLAKLENSEDLNLQFTKDSDDNVTLSFNKDGNTIGKTIIEKSSQFGTFSIGDGYYLDLNDGKFKYILSGESSNADLAQNTLHFNKGEELQVHAAAKDNQIILDHEHQHYINIYSNTQTNIKGFEVGKDKLQLSLLSNNLSSDTKLKFSGYDIEGGDVNITSGNTYITLSGAGHTDYASKTFNELVTMYLV

SEQ ID NO: 3 - protein, 949 aa, Gallibacterium anatis, strain 12656-12
  MISC_FEATURE (1)..(949): N-terminal part of GtxA polypeptide (amino acids 1-949 of full length GtxA)
MLSLKEKVTGIDFDAIKDKVVSLKNTVSNIDFNLVKEDISSLKSNALSIAASDFKNKPVLFKDSLDLLTDATNTLRKITNQMSSISEISNKSLDLLDSLFEAAKDIVNIAYSKGGVEITKSATELAAKAALIVDKSIILANKDNTISEAVYHSINNSLQNIQKTAINIATHSHNEDKAEIAKASFELLSQVSDVISNALKNSGDIGIESQLLADINQFSHSILNTAKTVTDIATMDMNDKTSIAKNSISLIANVNDVISDILVMTDKDTELLNAIHNVTAKNLQNIEESAVNLANADVLSQEGKVSIAINSLTLISQTNKIVAQVLNEANLSTDKTQFVGELTDVLLNTAKSITLLATGNNATTAGKEQLAVASTNLIGNVNDLIQSITSFKGKEDIGNALHSAVDGQLSQIKQLAVALSNSNLDSSQGKTAIAITSFGLIAQANNIINKFLDNMSLSTNVSKSVHSLTNSALDAAKILTNVVQVDANNNQGKVVIANSSLELSKTASDIVSTVLKSTSISTQHIDIIHNAVNKTLTEMKDSAVAIALASSENNSAEIATHSLSLLSDASNMLKDIMQGMSPNNVIAPKTLELFNSLFATAQNIVQLADAKSSENIAKASVDLVQSATIILNNVLTLANVDSSLSKAFHQSFDASVSQIKEVAAQLATASSASNKAEIAKLSFDFISQVSDLATNTLTTAKTGLDSTLLNNVNGLSHSVLNAAKSVTDIIVSDNPANTASLSVSLVNNANEIVSNILTLSGKQNTLSTAVHDVTAKHLAPIEKIAINLANADNSSSDGKVAIALNSLTLIAQSNHLIEEVLKEAKLDNAKSAFAHNLTDLVLDTAKTITALASADTSKVDGKQQIASASTHLVGQINEIVKSITTITNSETKVGNAAYQALKTHLEQVETIAVKLAAANASTAEGRTEIAIESFNLIAKTNGIMTDFLN

SEQ ID NO: 4 - DNA, 6078 bp, Gallibacterium anatis, strain 12656-12
  misc_feature (1)..(6078): Polynucleotide sequence coding for full length GtxA
gtgctttcattaaaagaaaaagtaactggaatagattttgatgcaatcaaagataaagtcgtttcattaaaaaacacggtttcaaatattgattttaatctggttaaagaagatatttcttctttaaaaagcaatgcgttatccatcgcggcatcagattttaaaaataaaccggtgttattcaaagactctttagacttacttactgatgctacaaatacactcagaaagattaccaatcaaatgtcatcaattagcgaaatttctaataagtcattagatttgctggattctctttttgaggctgccaaagatattgtaaacattgcctattcaaaaggtggtgtcgaaattactaagtctgcgacagaattagcggcaaaagcggcattaattgttgataaaagtatcatattagcaaataaagataatacaattagtgaagctgtttatcattctattaacaactcattacaaaatattcaaaaaacagctatcaatattgctacacattcacataatgaagataaagctgaaattgctaaagcctcttttgagctgttatctcaagttagtgatgttatcagtaatgcgttaaaaaattcaggtgatataggtatcgaatcacaactcttagccgatattaatcagttttctcattctattttgaacacagctaaaacagttactgatatagctactatggatatgaatgataaaacctcaatcgctaaaaatagcatttcattaatagccaatgtgaatgatgttatttccgatattctagtaatgacggataaagacaccgaattattaaatgcaattcataatgttactgcgaaaaatctacagaatatcgaagagagtgcggtcaatcttgcaaatgctgatgtgctgtctcaagaaggcaaagtcagtattgccattaattctttaactttaatatcacaaaccaacaaaattgttgcgcaagtgctaaatgaagctaatttaagcactgataaaacccaatttgttggcgaattaaccgatgtattattgaataccgcaaaaagtattacattgttagctaccggtaataatgcgacaacagcaggaaaggaacagctggcagttgcctcaaccaatcttattggtaacgtgaatgatctcattcaatcaattaccagctttaaaggcaaagaagatattggtaacgctttacacagtgcggtggacggacaattatcacaaatcaaacaacttgcggtcgcgttatcaaacagtaatcttgattcttcacaaggtaaaactgcaatagccatcacctctttcggcttgattgcacaagcaaataatattatcaataaattcttggataatatgagtttaagtactaatgtgagtaaatcggttcatagtttgactaattcagcgctagatgcagccaaaattctcacaaacgtagtacaagtagatgctaataacaatcaaggaaaggtcgtgattgccaatagttcattagaactttctaaaacagcaagtgatattgtgtctactgtgttaaaaagcacatctatttcaacacaacatattgatataattcataatgcagtaaataaaacattaacagaaatgaaagatagtgcggtagcaatagcacttgcttcatctgaaaataatagcgctgaaattgcaacgcattcattaagtctgttatccgatgcaagtaatatgttgaaagatattatgcaaggaatgagccctaataatgtcattgctccgaaaacattagaattatttaactcactatttgcgacagctcaaaatatcgttcaattagctgacgcaaaatcttcagaaaacattgctaaagctagtgttgatttggtacaaagcgcaacgattatcctcaataacgtattaacgttggctaacgttgattcttctttaagtaaagcttttcatcaatcatttgatgcttcagtttctcaaattaaagaggtagcagctcaattagctaccgcgtcttctgcctctaataaagctgagattgcaaaactctcttttgattttattagtcaagtaagtgatttagcgaccaacaccttaacaacagcgaaaaccggattagatagcacgctgctgaataacgttaacggtctttctcattccgtcttaaatgcagcaaaatcagtaaccgatattattgtgagtgataacccagcgaataccgccagtttatccgtttctttggtgaataatgccaatgaaattgtttcaaatatcttaaccttatccggaaaacaaaatacgctctccacggcagtacacgatgtaaccgctaaacatttagcgccgattgagaaaatagcaattaaccttgcgaatgccgataactcaagcagtgatggaaaagttgctattgcgttaaactcattaacattgattgcacaaagtaaccatttaatcgaagaagtattaaaagaggctaaattagataatgcgaagagcgcctttgctcacaatttaacggatttagtattagataccgccaaaacaatcacggcattagcatcagcggataccagtaaagtagacggcaagcagcagattgcctctgcatcaacacatttagtcggacaaattaatgagattgtcaaatcaatcacgacaataaccaattcagaaacgaaagtcggcaatgccgcatatcaagcgttaaaaacacatttagagcaggtagaaacaattgcggttaaacttgccgccgccaatgcatcaacagcggaaggcagaacagaaattgcgattgaatctttcaatttaatcgcaaaaaccaatggcataatgaccgatttcctaaatcaaatcggcataaaagaagagttaaccaaaccaatccaaggtttatctaattctattttagatactgccaaaaccttaacttatgttgtacaaattgacccaacaacagacaaaggtaaactttctatcgcagatagctcatttgaattggcaaaatctgctaaccaaattgtctcatatattatggatttatccggcagctcaagtgaactaagccataatattgcgaatacggctcatcaaatcttgtctatatcccaagacagactattaagcattggaaataatatttctgcattggcaaatgccgataaactcacaaaagaaggcgtgaaaattattgtagacagttcatttgccatcaccagcgatgtaaatggctttatcactgatgtggtgaaaacagtaggcaaagacggcaatcctaaagtggggtcagccctatcattatccaactccattattgatatgggacattctatcgcaaacctaattcaatcggatgttaatacaagtagcgggaaagcggctattgcagaaggatcaattaaattaattggcaatattaacggattagtcagcgatgtgttaagcctttctaatgcctctaccgccgtttctgaagctataagttcttctgcgggaggtattttaactaatttatcttctttgataggttcatcaattaaacttcataattggtctaatatgacccaagccgatcaaattgcagtggggtttgatattggattgaaagcggtaagcactattgcgacaggagttggcacaacagcacaatccattgcaaaaataattggtactactacgatgttgccacaaattggtgctgctgtatcaggaatcgctctggcagcaagtccgttagagataaaaggcttagttgacgaacataaatatgtaaaacaaattgattctcttgcatcagaaacaaaaacttatggttatcaaggtgatgaattattagccagccttttaaatgaaaaatttgcacttaataccgcatatacagctacagacattgctttaaatttagcaactacagcgatctctgtggcagcaacagcaagtgtcattggtgcaccgattgcggcaattgcaggtgtagtgagaggagctatcggcggtattatgtcggcgatcaaacaaccagcattggaacatattgctaaacgttatgtcgatcaaattgaaaagtatggtgatattcaaaaatactttgatcaaaatactgaggcaacattaaataaattctatgcgagtcaggaagtcattcaatcatttaagcaattacagaaattatataatgttgacaacattatcacccttgatggcgttgccagctcagacagtgcgatagaattagccgctattaccaaattagttgagcaaatgaataaagcaaataattatgctcaacttattcgtaacggtgaaatcgataaagctctcagcgctcaatatttgagtatggatgctaaaacgggcgtgttggaaattaccgcaccaggcaattcattaataaaatttaatagtcctctgtttgcgccgggtgttgaagaagcgcgcagaaaagcggttggcaaaaataatttttataccgatcttattattaatggtccaaatgaacatactattaatgacggcgcaggtaataatatttttatttctaatgataaatatgcctctgttttatatgacgaaaatggaaaattattgaagcatattaacctcaatatcaatgccggcgatggaaatgatacttatattgctgataacggacattctctatttaatggaggtaatggaacagatagtgtaagttataataatgaacatattcacggcattgttgttcatgggcgagatgccggtacttattctgtaacaaaacatattgctgatgcagaagtaactgttgaaaatattaaagtgaaaaatcatcaatacggtaaacgacaagaacgagttgagtatagagagttacatattgaaacaaaatcttatgatgcaagcgatatgctttacaacgtcgaagttatcagtgccagtgactatgatgatgttatgtatggtagtaaaggtaatgattacttcctcgcacaaaatggtaatgatcttgtttatggtaaagagggtgatgacattattttcggtggtgctggtgatgataaactttatggagaaaccggtaacgacaccttaaatggcggtttaggcaaagatttaatttatggtggcgaaggaaatgataccattattcaagatgatgctcttagttccgacactatcttcggcgatgaagggattgatacattagatcttcgtcatttagtgattaatgatgaaggactcggtgttgttgctgatctccagtccgagaagctttataaaggaaccatctttgatcatatctatgatatagagaatattataggtacatcaggaaatgataaccttattggaaatcataaagataatatcttaatcggtaatgatggtgatgatattttagaaggctatagcggtaatgatgtacttgccggaggaagtggcataaataaactttatggcggacagggagcagatatttatctcttatccaccaacgccacaaattatatcttcgatctgacaaaaaataatttagcaaaattagagaattcagaagatcttaaccttcaattcactaaagatagcgatgacaatgttaccttatctttcaataaagatggcaatactatcggtaaaaccattatagaaaaatcgagccaatttggcacattctcaataggtgatggatattacttagatcttaatgatggcaaatttaagtatattttatccggagaaagctccaatgccgatttagctcaaaatacattacattttaataaaggtgaagaacttcaagttcatgctgctgccaaggataaccaaattatattagatcacgaacatcagcattacattaatatttatagtaatacacagactaatattaaaggctttgaagttggtaaagataagctgcaattatcactgctcagtaataatttaagctcagacaccaaattaaaattcagcggttatgatattgaaggaggcgatgttaatattacttccggcaatacctatattacgttgtccggtgcggggcatacagattatgcgagcaaaacatttaatgaactggtcactatgtatcttgtt

SEQ ID NO: 5 - DNA, 3231 bp, Gallibacterium anatis, strain 12656-12
  misc_feature (1)..(3231): Polynucleotide sequence coding for N-terminally truncated GtxA polypeptide (nucleotides 2848-6078)
caaatcggcataaaagaagagttaaccaaaccaatccaaggtttatctaattctattttagatactgccaaaaccttaacttatgttgtacaaattgacccaacaacagacaaaggtaaactttctatcgcagatagctcatttgaattggcaaaatctgctaaccaaattgtctcatatattatggatttatccggcagctcaagtgaactaagccataatattgcgaatacggctcatcaaatcttgtctatatcccaagacagactattaagcattggaaataatatttctgcattggcaaatgccgataaactcacaaaagaaggcgtgaaaattattgtagacagttcatttgccatcaccagcgatgtaaatggctttatcactgatgtggtgaaaacagtaggcaaagacggcaatcctaaagtggggtcagccctatcattatccaactccattattgatatgggacattctatcgcaaacctaattcaatcggatgttaatacaagtagcgggaaagcggctattgcagaaggatcaattaaattaattggcaatattaacggattagtcagcgatgtgttaagcctttctaatgcctctaccgccgtttctgaagctataagttcttctgcgggaggtattttaactaatttatcttctttgataggttcatcaattaaacttcataattggtctaatatgacccaagccgatcaaattgcagtggggtttgatattggattgaaagcggtaagcactattgcgacaggagttggcacaacagcacaatccattgcaaaaataattggtactactacgatgttgccacaaattggtgctgctgtatcaggaatcgctctggcagcaagtccgttagagataaaaggcttagttgacgaacataaatatgtaaaacaaattgattctcttgcatcagaaacaaaaacttatggttatcaaggtgatgaattattagccagccttttaaatgaaaaatttgcacttaataccgcatatacagctacagacattgctttaaatttagcaactacagcgatctctgtggcagcaacagcaagtgtcattggtgcaccgattgcggcaattgcaggtgtagtgagaggagctatcggcggtattatgtcggcgatcaaacaaccagcattggaacatattgctaaacgttatgtcgatcaaattgaaaagtatggtgatattcaaaaatactttgatcaaaatactgaggcaacattaaataaattctatgcgagtcaggaagtcattcaatcatttaagcaattacagaaattatataatgttgacaacattatcacccttgatggcgttgccagctcagacagtgcgatagaattagccgctattaccaaattagttgagcaaatgaataaagcaaataattatgctcaacttattcgtaacggtgaaatcgataaagctctcagcgctcaatatttgagtatggatgctaaaacgggcgtgttggaaattaccgcaccaggcaattcattaataaaatttaatagtcctctgtttgcgccgggtgttgaagaagcgcgcagaaaagcggttggcaaaaataatttttataccgatcttattattaatggtccaaatgaacatactattaatgacggcgcaggtaataatatttttatttctaatgataaatatgcctctgttttatatgacgaaaatggaaaattattgaagcatattaacctcaatatcaatgccggcgatggaaatgatacttatattgctgataacggacattctctatttaatggaggtaatggaacagatagtgtaagttataataatgaacatattcacggcattgttgttcatgggcgagatgccggtacttattctgtaacaaaacatattgctgatgcagaagtaactgttgaaaatattaaagtgaaaaatcatcaatacggtaaacgacaagaacgagttgagtatagagagttacatattgaaacaaaatcttatgatgcaagcgatatgctttacaacgtcgaagttatcagtgccagtgactatgatgatgttatgtatggtagtaaaggtaatgattacttcctcgcacaaaatggtaatgatcttgtttatggtaaagagggtgatgacattattttcggtggtgctggtgatgataaactttatggagaaaccggtaacgacaccttaaatggcggtttaggcaaagatttaatttatggtggcgaaggaaatgataccattattcaagatgatgctcttagttccgacactatcttcggcgatgaagggattgatacattagatcttcgtcatttagtgattaatgatgaaggactcggtgttgttgctgatctccagtccgagaagctttataaaggaaccatctttgatcatatctatgatatagagaatattataggtacatcaggaaatgataaccttattggaaatcataaagataatatcttaatcggtaatgatggtgatgatattttagaaggctatagcggtaatgatgtacttgccggaggaagtggcataaataaactttatggcggacagggagcagatatttatctcttatccaccaacgccacaaattatatcttcgatctgacaaaaaataatttagcaaaattagagaattcagaagatcttaaccttcaattcactaaagatagcgatgacaatgttaccttatctttcaataaagatggcaatactatcggtaaaaccattatagaaaaatcgagccaatttggcacattctcaataggtgatggatattacttagatcttaatgatggcaaatttaagtatattttatccggagaaagctccaatgccgatttagctcaaaatacattacattttaataaaggtgaagaacttcaagttcatgctgctgccaaggataaccaaattatattagatcacgaacatcagcattacattaatatttatagtaatacacagactaatattaaaggctttgaagttggtaaagataagctgcaattatcactgctcagtaataatttaagctcagacaccaaattaaaattcagcggttatgatattgaaggaggcgatgttaatattacttccggcaatacctatattacgttgtccggtgcggggcatacagattatgcgagcaaaacatttaatgaactggtcactatgtatcttgtt

SEQ ID NO: 6 - DNA, 2847 bp, Gallibacterium anatis, strain 12656-12
  misc_feature (1)..(2847): Polynucleotide sequence coding for C-terminally truncated GtxA (nucleotides 1-2847)
gtgctttcattaaaagaaaaagtaactggaatagattttgatgcaatcaaagataaagtcgtttcattaaaaaacacggtttcaaatattgattttaatctggttaaagaagatatttcttctttaaaaagcaatgcgttatccatcgcggcatcagattttaaaaataaaccggtgttattcaaagactctttagacttacttactgatgctacaaatacactcagaaagattaccaatcaaatgtcatcaattagcgaaatttctaataagtcattagatttgctggattctctttttgaggctgccaaagatattgtaaacattgcctattcaaaaggtggtgtcgaaattactaagtctgcgacagaattagcggcaaaagcggcattaattgttgataaaagtatcatattagcaaataaagataatacaattagtgaagctgtttatcattctattaacaactcattacaaaatattcaaaaaacagctatcaatattgctacacattcacataatgaagataaagctgaaattgctaaagcctcttttgagctgttatctcaagttagtgatgttatcagtaatgcgttaaaaaattcaggtgatataggtatcgaatcacaactcttagccgatattaatcagttttctcattctattttgaacacagctaaaacagttactgatatagctactatggatatgaatgataaaacctcaatcgctaaaaatagcatttcattaatagccaatgtgaatgatgttatttccgatattctagtaatgacggataaagacaccgaattattaaatgcaattcataatgttactgcgaaaaatctacagaatatcgaagagagtgcggtcaatcttgcaaatgctgatgtgctgtctcaagaaggcaaagtcagtattgccattaattctttaactttaatatcacaaaccaacaaaattgttgcgcaagtgctaaatgaagctaatttaagcactgataaaacccaatttgttggcgaattaaccgatgtattattgaataccgcaaaaagtattacattgttagctaccggtaataatgcgacaacagcaggaaaggaacagctggcagttgcctcaaccaatcttattggtaacgtgaatgatctcattcaatcaattaccagctttaaaggcaaagaagatattggtaacgctttacacagtgcggtggacggacaattatcacaaatcaaacaacttgcggtcgcgttatcaaacagtaatcttgattcttcacaaggtaaaactgcaatagccatcacctctttcggcttgattgcacaagcaaataatattatcaataaattcttggataatatgagtttaagtactaatgtgagtaaatcggttcatagtttgactaattcagcgctagatgcagccaaaattctcacaaacgtagtacaagtagatgctaataacaatcaaggaaaggtcgtgattgccaatagttcattagaactttctaaaacagcaagtgatattgtgtctactgtgttaaaaagcacatctatttcaacacaacatattgatataattcataatgcagtaaataaaacattaacagaaatgaaagatagtgcggtagcaatagcacttgcttcatctgaaaataatagcgctgaaattgcaacgcattcattaagtctgttatccgatgcaagtaatatgttgaaagatattatgcaaggaatgagccctaataatgtcattgctccgaaaacattagaattatttaactcactatttgcgacagctcaaaatatcgttcaattagctgacgcaaaatcttcagaaaacattgctaaagctagtgttgatttggtacaaagcgcaacgattatcctcaataacgtattaacgttggctaacgttgattcttctttaagtaaagcttttcatcaatcatttgatgcttcagtttctcaaattaaagaggtagcagctcaattagctaccgcgtcttctgcctctaataaagctgagattgcaaaactctcttttgattttattagtcaagtaagtgatttagcgaccaacaccttaacaacagcgaaaaccggattagatagcacgctgctgaataacgttaacggtctttctcattccgtcttaaatgcagcaaaatcagtaaccgatattattgtgagtgataacccagcgaataccgccagtttatccgtttctttggtgaataatgccaatgaaattgtttcaaatatcttaaccttatccggaaaacaaaatacgctctccacggcagtacacgatgtaaccgctaaacatttagcgccgattgagaaaatagcaattaaccttgcgaatgccgataactcaagcagtgatggaaaagttgctattgcgttaaactcattaacattgattgcacaaagtaaccatttaatcgaagaagtattaaaagaggctaaattagataatgcgaagagcgcctttgctcacaatttaacggatttagtattagataccgccaaaacaatcacggcattagcatcagcggataccagtaaagtagacggcaagcagcagattgcctctgcatcaacacatttagtcggacaaattaatgagattgtcaaatcaatcacgacaataaccaattcagaaacgaaagtcggcaatgccgcatatcaagcgttaaaaacacatttagagcaggtagaaacaattgcggttaaacttgccgccgccaatgcatcaacagcggaaggcagaacagaaattgcgattgaatctttcaatttaatcgcaaaaaccaatggcataatgaccgatttcctaaat

SEQ ID NO: 7 - DNA, 28 bp, Artificial Sequence
  PCR primer sequence
tatcgtcgactatccatcgcggcatcag

SEQ ID NO: 8 - DNA, 31 bp, Artificial Sequence
  PCR primer sequence
agctgaattcaagcaagtgctattgctaccg

SEQ ID NO: 9 - DNA, 30 bp, Artificial Sequence
  PCR primer sequence
agctgaattcttatgtcggcgatcaaacaa

SEQ ID NO: 10 - DNA, 30 bp, Artificial Sequence
  PCR primer sequence
tatgtctagaggcgttggtggataagagat

SEQ ID NO: 11 - DNA, 20 bp, Artificial Sequence
  PCR primer sequence
cgatagattgtcgcacctga

SEQ ID NO: 12 - DNA, 19 bp, Artificial Sequence
  PCR primer sequence
tatggaactgcctcggtga

SEQ ID NO: 13 - DNA, 23 bp, Artificial Sequence
  PCR primer sequence
tgatgcaatcaaagataaagtcg

SEQ ID NO: 14 - DNA, 19 bp, Artificial Sequence
  PCR primer sequence
aatcggcattggagctttc

SEQ ID NO: 15 - DNA, 19 bp, Artificial Sequence
  PCR primer sequence
aaccaaaccaatccaaggt

SEQ ID NO: 16 - DNA, 20 bp, Artificial Sequence
  PCR primer sequence
attgccgtctttgcctactg

SEQ ID NO: 17 - DNA, 42 bp, Artificial Sequence
  PCR primer sequence
agtcccatgggtctttcattaaaagaaaaagtaactggaata

SEQ ID NO: 18 - DNA, 37 bp, Artificial Sequence
  PCR primer sequence
cagtctcgagttatgaattttcttctataaaagcagc

SEQ ID NO: 19 - DNA, 37 bp, Artificial Sequence
  PCR primer sequence
agtcccatggcaattgaatctttcaatttaatcgcaa

SEQ ID NO: 20 - DNA, 39 bp, Artificial Sequence
  PCR primer sequence
cagtctcgagttaatttaggaaatcggtcattatgccat

SEQ ID NO: 21 - DNA, 39 bp, Artificial Sequence
  PCR primer sequence
cagtctcgagttaaacaagatacatagtgaccagttcat

SEQ ID NO: 22 - DNA, 39 bp, Artificial Sequence
  PCR primer sequence
gttatccataataattaatttaggaaatcggtcattatg

SEQ ID NO: 23 - DNA, 37 bp, Artificial Sequence
  PCR primer sequence
ttcctaaattaattatggataacttctcaactttagg

SEQ ID NO: 24 - DNA, 21 bp, Artificial Sequence
  PCR primer sequence
caaacctaattcaatcggatg

SEQ ID NO: 25 - DNA, 23 bp, Artificial Sequence
  PCR primer sequence
tgcttcaataattttccattttc

SEQ ID NO: 26 - DNA, 30 bp, Artificial Sequence
  PCR primer sequence
ctgatctagacgccgtaaatcgcataatca

SEQ ID NO: 27 - DNA, 26 bp, Artificial Sequence
  PCR primer sequence
cgaattcccggcagaaaaggtcaaca

SEQ ID NO: 28 - DNA, 34 bp, Artificial Sequence
  PCR primer sequence
tttctgccgggaattcggcgaatggtgtgagaag

SEQ ID NO: 29 - DNA, 28 bp, Artificial Sequence
  PCR primer sequence
tcaagtcgacaagccaaagccaatacga

SEQ ID NO: 30 - DNA, 18 bp, Artificial Sequence
  PCR primer sequence
tttcctgtaatgcctgct

SEQ ID NO: 31 - DNA, 18 bp, Artificial Sequence
  PCR primer sequence
ttttgatcgttcgggctt

(19) United States
(12) Patent Application Publication
(10) Pub. No.: US 2012/0258515 A1
(43) Pub. Date: Oct. 11, 2012

(54) Cytolytic RTX-Toxin From Gallibacterium Anatis

(75) Inventors: Anders Miki Bojesen, Kobenhavn O (DK); Bodil Marie Kristensen, Kobenhavn O (DK)

(73) Assignee: KØbenhavns Universitet

(21) Appl. No.: 13/499,987

(22) PCT Filed: Oct. 7, 2010
(86) PCT No.: PCT/DK10/50257

§ 371 Date: Jun. 27, 2012

Related U.S. Application Data
(60) Provisional application No. 61/250,163, filed on Oct. 9, 2009.

(51) Int. Cl. C07K 14/285; C07K 19/00; C12N 9/96

(52) U.S. Cl. 435/188; 530/350

(57) ABSTRACT

The present invention relates to the field of animal health and in particular the causative agent of a new bacterial poultry disease caused by Gallibacteruim spp, including Gallibacterium anatis, Gallibacterium genomospecies 1 and Gallibacterium genomospecies 2. The invention provides a novel RTX toxin from said Gallibacterium species, the novel toxin being named GtxA (Gallibacterium toxin). In addition the invention provides the amino acid and nucleotide sequences of GtxA, a vaccine comprising inactivated toxoid or fragments of the toxoid as well as methods of immunizing birds to prevent said disease and to methods of diagnosing a Gallibacterium anatis infection in birds.

All patent and non-patent references cited in the application, or in the present application, are also hereby incorporated by reference in their entirety.

### FIELD OF INVENTION

The invention belongs to the field of animal health and in particular the causative agent of a new bacterial poultry disease caused by Gallibacteruim spp, including Gallibacterium anatis, Gallibacterium genomospecies 1 and Gallibacterium genomospecies 2. The invention provides a novel RTX toxin from said Gallibacterium species, the novel toxin being named GtxA (Gallibacterium toxin). In addition the invention provides the amino acid and nucleotide sequences of GtxA, a vaccine comprising inactivated toxoid or fragments of the toxoid as well as methods of immunizing birds to prevent said disease and to methods of diagnosing a Gallibacterium anatis infection in birds.

### BACKGROUND OF INVENTION

During the last decade, intensive poultry farming methods to increase productivity, has resulted in an increase of disease manifestation throughout all major poultry producing countries. This has caused an increasing need for new and better vaccines and vaccination programs to control these diseases. Nowadays, many animals are immunized against a number of diseases of viral and bacterial origin. Examples of viral diseases in poultry are Newcastle Disease, Infectious Bronchitis, Avian Pneumovirus, Fowlpox, Infectious Bursal Disease etc. Examples of bacterial diseases are Avian Coryza caused by Haemophilus paragallinarum (upper respiratory tract), Bordetella avium (upper respiratory tract), Ornithobacterium rhinotracheale (lower respiratory tract), Salmonella infections (digestive tract), Pasteurella multocida, which is the causative agent of fowl cholera (septicemic), and E. coli infections.

Inflammation in the reproductive organs and peritoneum of egg-layers is a recurrent problem in commercial egg-layer flocks causing egg production drop, increased mortality and consequential economical losses and lowered animal welfare. Avian pathogenic E. coli is often isolated from these lesions, but, several studies have demonstrated Gallibacterium anatis to be a frequent cause of oophoritis, salpingitis and peritonitis, either alone or as a co-pathogen. Moreover, G. anatis has been isolated from avian cases of septicaemia, hepatitis, enteritis and upper respiratory tract lesions. G. anatis is a common part of the normal flora of both the upper respiratory tract and lower genital tract of egg-laying hens and other avian species (Bojesen A. M., Nielsen S. S., Bisgaard M., Prevalence and transmission of haemolytic Gallibacterium species in chicken production systems with different biosecurity levels, Avian Pathol. (2003) 32:503-510), and can therefore be regarded as an opportunistic pathogen. Its pathogenesis has not been studied in depth, particularly not at the molecular level, and little is known about the genes and mechanisms behind G. anatis' ability to cause disease. G. anatis is divided into two biovars; the β-haemolytic biovar haemolytica and the non-haemolytic biovar anatis. The ability to lyse red blood cells is a prominent phenotype of pathogenic G. anatis isolates (Christensen H., Bisgaard M., Bojesen A. M., Mutters R., Olsen J. E., Genetic relationships among avian isolates classified as Pasteurella haemolytica, ‘Actinobacillus salpingitidis’ or Pasteurella anatis with proposal of Gallibacterium anatis gen. nov., comb. nov and description of additional genomospecles within Gallibacterium gen. nov, Int. J. Syst. Evol. Microbiol. (2003) 53:275-287). Gallibacterium is a Gram-negative genus belonging to the γ-proteobacterial family Pasteurellaceae (Christensen et al, op cit), and various pathogenic members of Pasteurellaceae, e.g. the cause of periodontal disease in humans, Aggregatibacter actinomycemcomitans, the causative agent of bovine shipping fever Mannheimia haemolytica, and the swine pathogen Actinobacillus pleuropneumoniae produce haemolysins and leukotoxins belonging to the group of RTX-toxins (repeat in toxin).

G. anatis vaccines consisting of inactivated or live, attenuated bacteria are available. However, these vaccines do not confer protection against secreted haemolytic proteins from this species.

### SUMMARY OF INVENTION

The purpose of the present invention was to examine G. anatis biovar haemolytica's interactions with eukaryotic cells and to identify and characterize the genes and proteins responsible for the haemolytic phenotype. The inventors found G. anatis to be highly cytotoxic towards avian macrophages, a trait likely to play a key role in pathogenesis. Furthermore, the inventors identified and characterised a new type of RTX-toxin responsible for the leukotoxic and haemolytic activity in G. anatis biovar haemolytica.

The present invention relates to GtxA polypeptides and polynucleotides from bacteria of the genus Gallibacterium, most preferably selected from the group of Gallibacterium anatis, Gallibacterium genomospecies 1 and Gallibacterium genomospecies 2.

In a first aspect, the invention relates to an isolated polypeptide, said polypeptide comprising an amino acid sequence selected from the group consisting of

### a) SEQ ID No. 1, 2 or 3;

b) a sequence variant of the amino acid sequence selected from the group consisting of SEQ ID No. 1, 2 and 3, wherein the variant has at least 70% sequence identity to said SEQ ID No.; and
c) a fragment consisting of a least 150 contiguous amino acids of any of a), wherein any amino acid specified in the chosen sequence is changed to a different amino acid, provided that no more than 30 of the amino acids in the sequence are so changed.

The polypeptide having the amino acid sequence described in SEQ ID NO 1 is the RTX toxin from G. anatis. The protein, named GtxA (Gallibacterium toxin), consists of 2026 amino acids (aa). This is twice the size of the classical pore-forming RTX-toxins. The C-terminal 1000 aa of GtxA is homologous to the RTX-toxins in other members of Pasteurelleceae, e.g. 38% sequence similarity to A. pleuropneumoniae ApxIA. In contrast, the N-terminal approximately 950 aa has no significant matches in the GenBank database, but contains eleven 57-aa repeats of unknown function.

The GtxA toxin has several utilities, including but not limited to use as a toxoid vaccine and diagnostic uses to reveal an existing immune response against G. anatis in birds in general and in poultry in particular.

In a further aspect the invention relates to an isolated polynucleotide, said polynucleotide comprising a nucleic acid sequence selected from the group consisting of

### a) SEQ ID No. 4, 5 or 6;

b) a sequence variant of the amino acid sequence selected from the group consisting of SEQ ID No. 4, 5 and 6, wherein the variant has at least 60% sequence identity to said SEQ ID No.;
c) a fragment consisting of a least 450 contiguous nucleotides of any of a), wherein any nucleic acid specified in the chosen sequence is changed to a different nucleic acid, provided that no more than 90 of the nucleic acids in the sequence are so changed;
d) a polynucleotide capable of hybridising, under high stringency, to a polynucleotide being complimentary to SEQ ID No. 4, 5 or 6;
e) a polynucleotide encoding the polypeptides of SEQ ID No. 1, 2 or 3;
f) a polynucleotide encoding a sequence variant of the amino acid sequence selected from the group consisting of SEQ ID No. 1, 2 and 3, wherein the variant has at least 70% sequence identity to said SEQ ID No.; and
g) a polynucleotide encoding a fragment consisting of a least 150 contiguous amino acids of any of SEQ ID No. 1, 2 or 3, wherein any amino acid specified in the chosen sequence is changed to a different amino acid, provided that no more than 30 of the amino acids in the sequence are so changed.

Furthermore, the invention relates to a vector comprising the polynucleotide of the invention.

In further aspects the invention relates to medical uses of the polypeptide of the invention, the polynucleotide of the invention and the vector of the invention.

Preferably the medical use is for the treatment, and/or prophylactic treatment of a disease, a disorder or any damage caused by a bacterial infection.

In one aspect the invention relates to use of the polypeptide and/or polynucleotide of the invention for the preparation of a medicament for the treatment and/or prophylactic treatment of a disease, a disorder or any damage caused by a bacterial infection.

In further aspects the invention relates to an isolated host cell transformed or transduced with the vector of the invention and to a packaging cell line capable of producing an infective virion of the invention.

Furthermore, the invention relates to an antibody capable of binding specifically to an isolated polypeptide having an amino acid sequence selected from the group consisting of

### a) SEQ ID No. 1, 2 or 3;

b) a sequence variant of the amino acid sequence selected from the group consisting of SEQ ID No. 1, 2 and 3, wherein the variant has at least 70% sequence identity to said SEQ ID No.; and
c) a fragment consisting of a least 150 contiguous amino acids of any of a), wherein any amino acid specified in the chosen sequence is changed to a different amino acid, provided that no more than 30 of the amino acids in the sequence are so changed.

These antibodies against GtxA can be used in diagnostic and therapeutic aspects.

In further aspects, the invention relates to method for inactivation of the isolated polypeptide of the invention.

Furthermore, the invention relates to a vaccine composition comprising the isolated polypeptide or the isolated polynucleotide, prepared as naked DNA or as a vector, of the invention, optionally together with one or more suitable adjuvant(s), excipient(s), emulsifier(s) or carrier(s).

In another aspect, the invention relates to a method of administering the vaccine of the invention to the avian species as described herein, wherein said vaccine is administered by intra muscular or subcutaneous injection, orally through e.g. food or water, aerosols, scarification e.g. in the foot or wing web, eye drops or by in-ovo administration.

In further aspects the invention relates to the polypeptide or polynucleotide of the invention for use as a diagnostic marker.

The invention also provides a method of diagnosing a pathogenic bacterial Gallibacterium infection in an avian species, said method comprising detection of the polypeptide of the invention, detection of an antibody against said polypeptide, or detection of a polynucleotide of the invention.

Preferably the pathogenic bacterium is from the genus Gallibacterium, more preferably selected from the group of Gallibacterium anatis, Gallibacterium genomospecies 1 and Gallibacterium genomospecies 2.

Also provided is a kit for detecting the presence of the polypeptide the invention, said kit comprising at least one binding protein capable of binding said polypeptide, said binding protein being linked to a solid support. Preferably said binding protein is an antibody.

In one aspect, the invention relates to a kit for detection of an antibody against the polypeptide of the invention wherein said kit comprises said polypeptide immobilized to a solid surface.

### DESCRIPTION OF DRAWINGS

FIG. 1: Haemolytic activity of G. anatis culture supernatants and expression of GtxA.

A. Growth and haemolytic activity of cell free culture supernatant of G. anatis 12656-12. An overnight culture was diluted 1:100 and growth (cell density measured by absorbance at 600 nm) and haemolytic activity in cell-free culture supernatant were recorded. The haemolytic activity of supernatant diluted 100-fold in BHI is shown. Extracellular proteins for western blotting (FIG. 5B) were harvested in parallel. The experiment was repeated three times, the level of haemolytic activity varied but the relative pattern was consistent.
B. Levels of GtxA determined by western blotting with ApxI-antiserum. Supernatants were harvested at the indicated time points, concentrated 100-fold as described in material and methods and separated by SDS-PAGE in a 3-8% gel prior to blotting. Extracellular proteins from ΔgtxA were harvested at (OD₆₀₀=2 (lane marked ΔgtxA). WC=whole cell lysate from wild-type, the cells were harvested five hrs. after inoculation. Size markers are indicated on the left. The experiment was repeated with the same result.

FIG. 2: Haemolytic activity and cytotoxicity of G. anatis 12656-12 wild type (wt) and the isogenic gtxA mutant (ΔgtxA).

A. β-haemolysis. The bacteria were streaked on BHI-agar plates with 5% bovine blood and incubated at 37° C. for 18 hours.
B. Light microscopy (100× magnifications) of HD11 cells after one hour incubation with saline (mock), wt or ΔgtxA. Bacteria were harvested in late exponential phase (OD₆₀₀=1) and added at an m.o.i of 10. C. Cytotoxicity quantified with LDH activity. HD11 cells were incubated with bacteria as described in 1B. The averages of three replicate wells are shown, bars represent the S.E.

FIG. 3

A. The genetic organisation of gtxA, gtxC and their flanking genes in G. anatis 12656-12. Arrows indicate open reading frames. A predicted transcriptional terminator is indicated downstream of gtxC.
B. Organisation of GtxA. K indicates conserved lysine residues (Lys1484 and Lys1607). The glycine aspartate-rich region (position 1640-1830) is marked.
C. Alignment of the 15 repeats in the N-terminal domain of GtxA, the alignment was generated with Radar [Heger A., Holm L., Rapid automatic detection and alignment of repeats in protein sequences, Proteins (2000) 41:224-237]. Numbers to the right indicate amino acid position in GtxA. Positions where the amino acid are identical (black) or similar (grey) in more than 50% of the repeats are marked.

FIG. 4: Cytotoxic activity of E. coli expressing GtxA.

A. β-haemolytic activity of E. coli ER2566 grown on LB-agar with 5% bovine blood and 0.1 mM IPTG, incubated at 30° C. T1SS: + or − indicates the presence or absence of plasmid pLG575 which expresses the E. coli T1SS-components HIyB and HIyD. RTX=amino acids 931-2026 of GtxA, N-term=amino acids 1-949 of GtxA.
B. Liquid haemolysis assay and LDH cytotoxicity assay with E. coli ER2566/pLG575 expressing different versions of GtxA.

FIG. 5: Expression of GtxA in E. coli.

Western blot on whole cell lysate (WC) and extracellular protein (EC) from E. coli ER2566 after induction with IPTG. Proteins were separated by SDS-PAGE in a 4-12% gel and blotted on a PVDF-membrane. The blot was probed with ApxI-antiserum. B=blank, size markers are indicated on the right (PageRuler Prestained Protein Ladder Plus (Fermentas)). The upper band in each lane has the expected size of full length GtxA (215 kDa) or the RTX-domain (117 kDa). The smaller sized bands are likely degradation products.

FIG. 6: Alignment of amino acid sequences from various Gallibacterium strains. Amino acids in positions 1133-1515 of GtxA (SEQ ID No 1), are aligned versus the amino acid sequences of other toxins derived from other Gallibacterium strains. Dots indicate identical residues, whereas unconserved positions are highlighted.

FIG. 7

Culture supernatants from various G. anatis strains and the type strains of G. genomospecies 1 (CCM5974) and G. genomospecies 2 (CCM5976) were harvested at OD₆₀₀=0.6 and filter-sterilized. Extracellular proteins were precipitated as described in Methods and separated on a 3-8% Tris-Acetate SDS-gel, blotted onto a PVDF membrane and probed with ApxIA-antiserum. GtxA (215 kDa) is marked with an arrow. The larger bands are either an unidentified protein unrelated to GtxA (see FIG. 3) or modified versions of GtxA. M=molecular size marker (Spectra Multicolor High Range Protein Ladder (Fermentas)), sizes are indicated to the right (kDa).

FIG. 8: β-haemolytic activity and subcellular localisation of GtxA in a T1SS-mutant.

A. β-haemolysis of G. anatis 12656-12 and isogenic mutants. Photo of a blood agar plate with streaks of 12656-12 (wt), gtxA- and gtxBD mutants after incubation at 37° C. for 24 hours. Bacteria have been removed from the left part of the streak to reveal haemolysis under the colony. B. Subcellular localisation of GtxA. Cells and supernatant were harvested at the transition to stationary phase (OD₆₀₀=1.7). Extracellular proteins were precipitated as described in Methods. Whole cell lysates (pellet) and extracellular proteins (supernatant) were separated on a 3-8% Tris-Acetate SDS-gel and blotted onto PVDF membrane and probed with ApxIA-antiserum. GtxA is marked with an arrow. The identity was verified by mass spectrophotometry The antiserum additionally recognises an unidentified protein of approx. 270 kDa present in both wild-type and mutant strains. Size markers are indicated on the left (Spectra Multicolor High Range Protein Ladder (Fermentas)).

FIG. 9: Organisation of the gtxAC and gtxEBD loci in G. anatis 12656-12.

Arrows represent open reading frames (ORF). The position of primer pairs used for detection of gtxA and gtxEBD are indicated above each ORF with small arrows. The organisation of the typical RTX-toxin loci (Frey & Kuhnert 2002) is included for comparison.

### DEFINITIONS

The term ‘adjuvant’ used herein refers to a substance whose admixture with an administered immunogenic determinant/antigen/nucleic acid construct increases or otherwise modifies the immune response to said determinant.

The term ‘allelic variant’ used herein refers to an alternative form of the gene encoding SEQ ID No. 1. Alleles may result from at least one mutation in the nucleic acid sequence and may result in altered mRNAs or polypeptides whose structure or function may or may not be altered. Common mutational changes which give rise to alleles are generally ascribed to natural deletions, additions, or substitutions of nucleotides. Each of these types of changes may occur alone, or in combination with the others, one or more times in a given sequence.

The term ‘antibody’ used herein refers to an immunoglobulin molecules and active portions of immunoglobulin molecules. Antibodies are for example intact immunoglobulin molecules or fragments thereof retaining the immunologic activity.

The term ‘antigen’ used herein refers to a substance that can bind to a clonally distributed immune receptor (T-cell or B-cell receptor); usually a peptide, polypeptide or a multimeric polypeptide. Antigens are preferably capable of eliciting an immune response.

The term ‘binding assay’ used herein refers to any biological or chemical assay in which any two or more molecules bind, covalently or non-covalently, to each other thereby enabling measuring the concentration of one of the molecules.

The term ‘biological sample’ used herein refers to any sample selected from the group, but not limited to, serum, plasma, whole blood, saliva, urine, lymph, a biopsy, semen, faeces, tears, sweat, milk, cerebrospinal fluid, ascites fluid, synovial fluid.

The term ‘carrier’ used herein refers to an entity or compound to which antigens are coupled to aid in the induction of an immune response.

The term ‘conservative amino acid substitution’ defined herein refers to a substitution by which one amino acid is substituted for another with one or more shared chemical and/or physical characteristics. Amino acids may be grouped according to shared characteristics. A conservative amino acid substitution is a substitution of one amino acid within a predetermined group of amino acids for another amino acid within the same group, wherein the amino acids within a predetermined groups exhibit similar or substantially similar characteristics.

The term ‘detection moiety’ used herein refers to a specific part of a molecule, preferably but not limited to be a protein, able to bind and detect another molecule.

The term ‘diagnostic marker’ used herein refers to the characteristic of a compound, such as a protein, that can be used to determine which disorder an individual is suffering from.

The term ‘disorder’ used herein refers to a disease or medical problem, and is an abnormal condition of an organism that impairs bodily functions, associated with specific symptoms and signs. It may be caused by external factors, such as invading organisms, or it may be caused by internal dysfunctions.

The term ‘fragment’ used herein refers to a non-full length part of a nucleic acid or polypeptide. Thus, a fragment is itself also a nucleic acid or polypeptide, respectively.

The term ‘immunogenic’ used herein refers to the ability of a particular substance, such as an antigen or epitope, to provoke an immune response, wherein said immune response may be cellular or humoral.

The term ‘medicament’ used herein refers to a pharmaceutical drug, also referred to as medicine or medication, that can be loosely defined as any chemical substance, preferably a vaccine, intended for prophylactic, curative, ameliorative or symptomatic use. It is to be understood from the above, that the intended use of the present invention does not necessarily comprise 100% prevention, cure or amelioration of any disease but also partial prevention, cure or amelioration.

The term ‘pathogenicity’ as used herein refers to the ability of a pathogen, such as a microorganuism, such as Gallibacterium anatis, to produce an infectious disease in an organism.

The term ‘plasmid’ used herein refers to an extra-chromosomal DNA molecule separate from the chromosomal DNA which is capable of replicating independently of the chromosomal DNA.

The term ‘polynucleotide’ used herein refers to an organic polymer molecule composed of nucleotide monomers covalently bonded in a chain, e.g. DNA (deoxyribonucleic acid) and RNA (ribonucleic acid).

The term ‘polypeptide’ used herein refers to an organic compound, also known as a protein, which is a peptide having at least, and preferably more than two amino acids. The generic term amino acid comprises both natural and non-natural amino acids any of which may be in the ‘D’ or ‘L’ isomeric form.

The term ‘prophylactic treatment’ used herein refers to any medical procedure whose purpose is to prevent, rather than treat or cure a disease. The term preventing is not intended to be absolute and also includes partial prevention of the disease or of one or more symptoms of the disease.

The term ‘promoter’ used herein refers to a binding site in a DNA chain at which RNA polymerase binds to initiate transcription of messenger RNA by one or more nearby structural genes.

The term ‘RTX toxin’ (repeats in the structural toxin) used herein refers to a pore-forming protein toxins produced by a broad range of pathogenic Gram-negative bacteria.

The term ‘sequence identity’ used herein refers to the determination of percent identity between two sequences and can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN and BLASTP programs of Altschul, et al. (1990) J. Mol. Biol. 215:403-410.

In order to characterize the identity, subject sequences are aligned so that the highest order homology (match) is obtained. Based on these general principles, the “percent identity” of two nucleic acid sequences may be determined using the BLASTN algorithm [Tatiana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site (http://www.ncbi.nlm.nih.gov), and using the default settings suggested here (i.e. Reward for a match=1; Penalty for a mismatch=−2; Strand option=both strands; Open gap=5; Extension gap=2; Penalties gap x_dropoff=50; Expect=10; Word size=11; Filter on). The BLASTN algorithm determines the % sequence identity in a range of overlap between two aligned nucleotide sequences. As Blast is a Local alignment is it best suited for calculating the percent sequence identity in a range of overlap between two related sequences of different length.

Another preferred, non-limiting example of a mathematical algorithm utilized for the comparison of sequences is the CLUSTAL W (1.7) alignment algorithm (Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680.). CLUSTAL W can be used for multiple sequence alignment preferably using BLOSUM 62 as scoring matrix. When calculating sequence identities, CLUSTAL W includes any gaps made by the alignment in the length of the reference sequence. Sequence identities are calculated by dividing the number of matches by the length of the aligned sequences with gaps.

The term ‘signal peptide’ used herein refers to a short sequence of amino acids that determine the eventual location of a protein in the cell, also referred to as sorting peptide.

The term ‘toxin’ used herein refers to a poisonous substance produced by living cells or organisms that are capable of causing disease on contact with or absorption by body tissues.

The term ‘toxoid’ used herein refers to a bacterial toxin (usually an exotoxin) whose toxicity has been weakened or suppressed either by chemical or heat treatment, while other properties, e.g. immunogenicity, are maintained.

The term ‘transcription factor’ used herein refers to a protein that binds to specific DNA sequences and thereby controls the transfer of genetic information from DNA to mRNA.

The term ‘vaccine’ used herein refers to a substance or composition capable of inducing an immune response in an animal. An immune response being an immune response (humoral/antibody and/or cellular) inducing memory in an organism, resulting in the infectious agent, being met by a secondary rather than a primary response, thus reducing its impact on the host organism.

The term ‘vector’ used herein refers to a DNA molecule used as a vehicle to transfer foreign genetic material into another cell.

### DETAILED DESCRIPTION OF THE INVENTION

It is a major objective of the present invention to provide a vaccine composition comprising a specific RTX toxin, GtxA, from Gallibacterium anatis or an immunogenic variant or fragment hereof for use as a medicament in the treatment and/or prophylactic treatment of any disease caused by a bacterial infection with bacteria from the Gallibacterium genus in birds.

Gallibacterium anatis is part of the normal bacterial flora in the upper respiratory- and lower genital tract of chickens and other avian species. However, G. anatis has also been isolated from pathological lesions and is therefore considered to be a potential pathogen. The invention provides a novel virulence factor; a G. anatis RTX-toxin with an atypical organisation and a broad target cell range.

Cell-free, filter-sterilised supernatant from G. anatis cultures lysed both erythrocytes and avian-derived macrophage-like cells (HD11), indicating production of one or more exotoxins. In the genome sequence of G. anatis 12656-12, we identified an RTX-toxin gene. The encoded protein, named GtxA (Gallibacterium toxin), consisted of 2026 amino acids (aa). This is twice the size of the classical pore-forming RTX-toxins. The C-terminal 1000 aa of GtxA was homologous to the RTX-toxins in other members of Pasteurelleceae, e.g. 38% sequence similarity to A. pleuropneumoniae ApxIA. In contrast, the N-terminal approximately 950 aa had no significant matches in the Gen Bank database, but contained eleven 57-aa repeats of unknown function. E. coli expressing gtxA and its acetyltransferase activator, gtxC, became haemolytic and leukotoxic. The function of various truncated versions of GtxA was examined. The C-terminal RTX-domain displayed lower haemolytic activity than the intact toxin, indicating that the N-terminal domain was not essential but required for maximal hemolytic activity. Cytotoxicity towards HD11 cells was not detected with the C-terminal alone, suggesting the novel N-terminal repeat-domain to be essential for the cytotoxic effect towards leukocytes.

G. anatis' expression of gtxA was examined with western and northern blotting. GtxA was detected in the extracellular protein fraction in a growth phase-dependent manner, but was not detected in the cell-associated protein fraction, consistent with the predicted secretion of the toxin.

Eleven genotypically and phenotypically diverse Gallibacterium strains were examined for the presence and expression of gtxA, GtxA secretion levels, and the lytic activities of culture supernatants. gtxA was widely distributed and was found in all strains tested, including Gallibacterium genomospecies 1 and 2 (FIG. 6, alignment). Expression varied substantially among the strains, and the avirulent non-haemolytic type strain, F149^{T}, expressed diminutive amounts. GtxA levels in the supernatant correlated to some extent with levels of haemolytic activity and cytotoxic activity towards HD11 cells.

We expect GtxA to contribute significantly to the pathogenicity of G. anatis.

### GtxA

Not previously been described, GtxA is a large polypeptide of 2026 amino acids with the weight of 215 kDa, having SEQ ID No. 1. It may be divided into a C-terminal and an N-terminal fragment. An exemplary C-terminal fragment consists of the 1,077 amino acids (SEQ ID No. 2) resembling a classical RTX toxin featuring six tandemly repeated nonapeptides. An exemplary N-terminal consists of 949 amino acids (SEQ ID No. 3) being relatively hydrophobic and sharing little sequence similarity to other RTX toxins or other proteins in GenBank. The toxin activity depends on an activator, GtxC, which promotes fatty acid acylation of GtxA, i.e. toxicity depends on posttranscriptional acylation of the polypeptide. The non-acylated protein is not toxic.

GtxA displays a cytolytic phenotype, being mainly haemolytic and leukotoxic. The C-terminal RTX-domain displays lower haemolytic activity than the intact toxin, indicating that the N-terminal domain is not essential but required for maximal hemolytic activity. Cytotoxicity towards avian-derived macrophage-like HD11 cells was not detected with the C-terminal alone, suggesting the novel N-terminal repeat-domain to be essential for the cytotoxic effect towards leukocytes.

Gallibacterium strains from different geographical regions (Denmark, Czech Republic and Mexico) were screened for the presence of the gtxA gene, which was found in all strains tested, although with substantial variation in expression between individual strains. This variation appeared to be unrelated to geographical origin.

G. anatis is a part of the normal bacterial flora in the upper respiratory- and lower genital tracts of chickens, egg-laying hens and other avian species. However, G. anatis has also been isolated from avian pathological lesions, and G. anatis is believed to play a significant role to the pathogenesis in poultry.

It is thus an object of the present invention to provide a toxoid vaccine derived from the GtxA protein or an immunologically active polypeptide variant hereof for use as a medicament for the treatment and/or prevention of a disease. Said disease may result from a bacterial infection in a warm blooded animal, and it is a further object of the present invention to prevent or treat said disease. Another aspect of the invention relates to a polynucleotide encoding the GtxA protein or polynucleotides encoding an immunologically active polypeptide variant for the treatment and/or prevention of a disease.

GtxA polypeptides are highly conserved across different Gallibacterium anatis isolates as demonstrated by the alignment of partial amino acid sequences in FIG. 6. It is therefore expected that a vaccine composition comprising a GtxA toxoid will be effective against a high number of different G. anatis isolates and even against related species of the Gallibacterium genus.

### Bacterial Species

The present invention relates to polypeptides, polynucleotides and polynucleotide-carrying expression vectors. In one embodiment the polynucleotides and polypeptides are derived from Gallibacterium anatis. Additionally, the present invention also covers GtxA polypeptides and polynucleotides from bacteria of the Pasteurellaceae family, more preferably from the genus Gallibacterium, most preferably selected from the group of Gallibacterium anatis, Gallibacterium genomospecies 1 and Gallibacterium genomospecies 2.

GtxA has turned out to differ widely from other RTX toxins. By the molecular tools provided herein, the inventors have made the cloning or probing of further GtxA-like toxins from the Gallibacteruim genus possible.

Such GtxA-like toxins from related species are expected to exhibit a certain degree of sequence homology to SEQ ID NO 1, 2 or 3 and the coding sequences are expected to hybridise to probes based on polynucleotides of the present invention.

### Genetically Modified Strains

As described in example 2, a G. anatis gtxA mutant strain has been produced by the present inventors. The strain was designated ΔgtxA. Example 7 describes an infection trial with such a G. anatis ΔgtxA mutant wherein birds infected with the wild type microorganism generally developed a disseminated and purulent inflammation involving the reproductive tract and the peritoneum, corresponding to lesions observed from natural infections with G. anatis in the field. Birds infected with the ΔgtxA mutant on the other hand generally developed a milder inflammation localized to the ovary. Accordingly it has been demonstrated that gtxA contributes substantially in the pathogenesis of G. anatis in chicken. It can thus be concluded that the present inventors have demonstrated that the ΔgtxA strain defined herein above can be used to immunize organisms. Such immunized organisms, e.g. an avian species, can thus develop immunity to a wild-type microorganism expressing gtxA, through antibodies generated against non-gtxA antigens e.g. on the surface of a specific pathogenic microorganism which microorganism is of the same species as the microorganism in which the gtxA expression and/or secretion has been abolished.

Thus, in one aspect, the present invention relates to a transgenic knock-out microorganism in which the endogenous gtxA genes have been disrupted to abolish expression of a functional gtxA polypeptide, and wherein said microorganism exhibits a reduced pathogenicity relative to a non-transgenic control microorganism.

In one embodiment, the microorganism is Gallibacterium anatis.

In a further embodiment, the transgenic microorganism does not possess antibiotic resistance.

### GtxA Polypeptides

One wild-type GtxA i.e. a naturally occurring non-mutated version of the protein is identified in SEQ ID No. 1.

In one aspect, the invention relates to an isolated polypeptide, said polypeptide comprising an amino acid sequence selected from the group consisting of

### a) SEQ ID No. 1, 2 or 3;

b) a sequence variant of the amino acid sequence selected from the group consisting of SEQ ID No. 1, 2 and 3, wherein the variant has at least 70% sequence identity to said SEQ ID No.; and
c) a fragment consisting of a least 150 contiguous amino acids of any of a), wherein any amino acid specified in the chosen sequence is changed to a different amino acid, provided that no more than 30 of the amino acids in the sequence are so changed.

Further wildtype GtxA polypeptides can be isolated from other Gallibacterium species and from other isolates of Gallibacterium anatis. These GtxAs are expected to share a high degree of sequence identity to SEQ ID NO 1, 2, and/or 3, as indicated in the alignment of fragments in FIG. 6.

In a preferred embodiment, the present invention relates to SEQ ID No. 1 and sequence variants of GtxA comprising a sequence identity of at least 70% to SEQ ID No. 1, more preferably 75% sequence identity, for example at least 80% sequence identity, such as at least 85% sequence identity, for example at least 90% sequence identity, such as at least 95% sequence identity, for example at least 96% sequence identity, such as at least 97% sequence identity, for example at least 98% sequence identity, such as at least 99% sequence identity, for example at least 99.5% sequence identity, such as at least 99.9% sequence identity with the GtxA sequence.

In another preferred embodiment, the present invention relates to the C-terminal domain of the GtxA polypeptide, which is defined in SEQ ID No. 2, and to sequence variants comprising a sequence identity of at least 70% to SEQ ID No. 2, more preferably 75% sequence identity, for example at least 80% sequence identity, such as at least 85% sequence identity, for example at least 90% sequence identity, such as at least 95% sequence identity, for example at least 96% sequence identity, such as at least 97% sequence identity, for example at least 98% sequence identity, such as at least 99% sequence identity with the C-terminal domain of the GtxA sequence.

In another preferred embodiment, the present invention relates to the N-terminal domain of the GtxA polypeptide, which is defined in SEQ ID No. 3, and to sequence variants comprising a sequence identity of at least 70% to SEQ ID No. 3, more preferably 75% sequence identity, for example at least 80% sequence identity, such as at least 85% sequence identity, for example at least 90% sequence identity, such as at least 95% sequence identity, for example at least 96% sequence identity, such as at least 97% sequence identity, for example at least 98% sequence identity, such as at least 99% sequence identity with the N-terminal domain of the GtxA sequence.

In addition to full-length GtxA, the present invention relates to fragments of GtxA. For example the C-terminal GtxA domain and to the N-terminal GtxA domain. Additionally, the present invention relates to fragments of these polypeptides. In a preferred embodiment, said fragments consists of at least 150 contiguous amino acids, preferably at least 200 amino acids more preferably at least 300 amino acids, more preferably at least 500 amino acids, more preferably at least 750 amino acids, more preferably at least 1000 amino acids, more preferably 1250 amino acids, more preferably at least 1500 amino acids, more preferably at least 1750 amino acids, more preferably at least 2000 amino acids.

The GtxA fragments may differ at one or more positions from the wildtype GtxA sequences. In a preferred embodiment, said fragments may contain up to 30 amino acid substitutions, more preferably up to 25 substitutions, more preferably up to 20 substitutions, more preferably up to 15 substitutions, more preferably up to 10 substitutions, more preferably up to 5 substitutions such as at four, three, two or one substitutions.

Other variants covered by the present invention relates to variants of the polypeptide of SEQ ID No. 1, 2 and 3, wherein conservative amino acid substitutions have occurred. In a preferred embodiment, the present invention relates to any polypeptide comprising SEQ ID No. 1, 2 or 3, wherein any amino acid in the polypeptide sequence has been conservatively substituted with another amino acid.

In another preferred embodiment, said sequence variants and fragments are immunogenic. In yet another preferred embodiment, said sequence variants and fragments retain biological activity, such as toxicity, wherein said toxicity comprises the formation of pores in the cellular membrane of the donee, for example cytotoxicity, such as cytolytic cytotoxicity, for example haemolytic cytotoxicity.

The present invention also relates to the polypeptides of SEQ ID No, 1, 2 and 3, wherein said polypeptides have been specifically modified as to remove the biological activity, such as for example toxicity, but keep activity such as for example immunogenicity. Therefore, in a preferred embodiment, the polypeptides of SEQ ID No. 1, 2 and 3 have been have been inactivated, preferably by heat or radiation, more preferably by being expressed in a non-acylated form, more preferably by exposure to a chemical substance such as for example formaldehyde.

In another preferred embodiment, the present invention relates to any polypeptide comprising SEQ ID No. 1, 2 or 3, wherein the signal peptide has been replaced by a heterologous signal peptide.

For purposes of purification, the present invention may be tagged. In a preferred embodiment, SEQ ID No. 1, 2 and 3 may be tagged with an affinity tag, preferably a cleavable tag such as a polyHis tag, for example a HA tag, such as a FLAG tag, for example a C-myc tag, such as a HSV tag, for example a V5 tag, such as a maltose binding protein tag, for example a cellulose binding domain tag, such as a BCCP tag, for example a Calmodulin tag, such as a Nus tag, for example a Glutathione-S-transferase tag, such as a Green fluorescent protein tag, for example a Thioredoxin tag, such as a S tag, for example a Strep tag.

Preferably, the tag is in the C-terminal portion of the protein, such as at the very C-terminal. More preferably, the tag is cleavable from the GtxA polypeptide by having a protease cleavage site inserted between the tag and the RTX polypeptide.

### GtxA Polynucleotides

The specific polynucleotide sequences are provided for by the present invention in SEQ ID No. 4, 5 and 6.

The present invention relates to an isolated polynucleotide, said polynucleotide comprising a nucleic acid sequence selected from the group consisting of

### a) SEQ ID No. 4, 5 or 6;

b) a sequence variant of the polynucleotides selected from the group consisting of SEQ ID No. 4, 5 and 6, wherein the variant has at least 60% sequence identity to said SEQ ID No.;
c) a fragment consisting of a least 450 contiguous nucleotides of any of a), wherein any nucleic acid specified in the chosen sequence is changed to a different nucleic acid, provided that no more than 90 of the nucleic acids in the sequence are so changed;
d) a polynucleotide capable of hybridising, under high stringency, to a polynucleotide being complimentary to SEQ ID No. 4, 5 or 6;
e) a polynucleotide encoding the polypeptides of SEQ ID No. 1, 2 or 3;
f) a polynucleotide encoding a sequence variant of the amino acid sequence selected from the group consisting of SEQ ID No. 1, 2 and 3, wherein the variant has at least 70% sequence identity to said SEQ ID No.; and
g) a polynucleotide encoding a fragment consisting of a least 150 contiguous amino acids of any of SEQ ID No. 1, 2 or 3, wherein any amino acid specified in the chosen sequence is changed to a different amino acid, provided that no more than 30 of the amino acids in the sequence are so changed.

Suitable experimental conditions for determining hybridization between a nucleotide probe and a homologous DNA or RNA sequence, involves pre-soaking of the filter containing the DNA fragments or RNA to hybridize in 5×SSC [Sodium chloride/Sodium citrate; cf. Sambrook et al.; Molecular Cloning: A Laboratory Manual, Cold Spring Harbor Lab., Cold Spring Harbor, N.Y. 1989] for 10 minutes, and pre-hybridization of the filter in a solution of 5×SSC, 5×Denhardt's solution [cf. Sambrook et al.; Op cit.], 0.5% SDS and 100 μg/ml of denatured sonicated salmon sperm DNA [cf. Sambrook et al.; Op cit.], followed by hybridization in the same solution containing a concentration of 10 ng/ml of a random-primed [Feinberg A P & Vogelstein B; Anal. Biochem. 1983 132 6-13], 32P-dCTP-labeled (specific activity >1×109 cpm/μg) probe for 12 hours at approximately 45° C. The filter is then washed twice for 30 minutes in 0.1×SSC, 0.5% SDS at a temperature of at least at least 60° C. (medium stringency conditions), preferably of at least 65° C. (medium/high stringency conditions), more preferred of at least 70° C. (high stringency conditions), and even more preferred of at least 75° C. (very high stringency conditions). Molecules to which the oligonucleotide probe hybridizes under these conditions may be detected using a x-ray film.

In one embodiment the nucleic acid molecule differs by a single nucleotide from a nucleic acid sequence selected from the group consisting of SEQ ID No. 4, 5, and 6. A single substitution may be a silent mutation or may give rise to a conservative amino acid substitution. A single substitution or deletion may also give rise to a frameshift mutation. In a more preferred embodiment, the invention relates to a polynucleotide sequence having at least 60% sequence identity to the polynucleotide of SEQ ID No. 4, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98%, more preferably at least 99% sequence identity with SEQ ID No. 4.

In another preferred embodiment, the invention relates to a polynucleotide sequence having at least 60% sequence identity to the polynucleotide of SEQ ID No. 5, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98%, more preferably at least 99% sequence identity with SEQ ID No. 5.

In another preferred embodiment, the invention relates to a polynucleotide sequence having at least 60% sequence identity to the polynucleotide of SEQ ID No. 6, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98%, more preferably at least 99% sequence identity with SEQ ID No. 6.

The present invention also relates to fragments of the polynucleotides of SEQ ID No. 1, 2 and 3. In a preferred embodiment, said fragments consisting of at least 450 contiguous nucleotides, more preferably at least 500 contiguous nucleotides, more preferably at least 600 contiguous nucleotides, more preferably at least 750 contiguous nucleotides, more preferably at least 1000 contiguous nucleotides, more preferably at least 1500 contiguous nucleotides, more preferably at least 2000 contiguous nucleotides, more preferably at least 2500 contiguous nucleotides, more preferably at least 3000 contiguous nucleotides, more preferably at least 3500 contiguous nucleotides, more preferably at least 4000 contiguous nucleotides, more preferably at least 4500 contiguous nucleotides, more preferably at least 5000 contiguous nucleotides, more preferably at least 5500 contiguous nucleotides, more preferably at least 6000 contiguous nucleotides.

The polynucleotide fragments may differ at one or more positions from the wildtype GtxA polynucleotide sequences, wherefrom said fragments are derived from. In a preferred embodiment, said fragments may contain up to 90 nucleotide substitutions, more preferably up to 80 substitutions, more preferably up to 70 substitutions, more preferably up to 60 substitutions, more preferably up to 50 substitutions, more preferably up to 40 substitutions, more preferably up to 30 substitutions, more preferably up to 20 substitutions, more preferably up to 10 substitutions more preferably up to 5 substitutions such as at four, three, two or one substitutions.

The present invention relates to a polynucleotide capable of hybridising to a polynucleotide having the sequence of SEQ ID No. 4, 5 and 6, preferably under high stringency hybridising conditions.

The polynucleotide of the present invention may comprise the nucleotide sequence of a naturally occurring allelic nucleic acid variant.

The polynucleotide of the present invention may also comprise a variant of SEQ ID No. 4, 5 and 6, wherein said polynucleotide has been optimized for expression in Escherichia coli.

### Expression Vectors

The polynucleotides of the invention may be comprised within any suitable vector, such as an expression vector or a cloning vector. Numerous vectors are available and any vector suitable the specific purpose may be selected. The appropriate nucleic acid sequence may be inserted into the vector by a variety of procedures, for example, DNA may be inserted into an appropriate restriction endonuclease site(s) using techniques well known in the art. Apart from the nucleic acid sequence relating to the invention, the vector may furthermore comprise one or more of a signal sequence, an origin of replication, one or more marker genes, an enhancer element, a promoter, and a transcription termination sequence. The vector may also comprise additional sequences, such as enhancers, poly-A tails, linkers, polylinkers, operative linkers, multiple cloning sites (MCS), STOP codons, internal ribosomal entry sites (IRES) and host homologous sequences for integration or other defined elements. The vector is preferably an expression vector, comprising the nucleic acid operably linked to a regulatory nucleic acid sequence directing expression thereof in a suitable cell.

In a preferred embodiment, the vector of the present invention is a plasmid vector, such as a eukaryotic plasmid vector, more preferably a prokaryotic plasmid vector.

In another preferred embodiment, the vector may also be a viral vector, preferably derived from the Retroviridae family, such as lentivirus, for example HIV, such as SIV, for example EAIV, such as CIV.

In yet a preferred embodiment, the vector may be selected from, but not limited to, the group comprising alphavirus, adenovirus, adeno associated virus, baculovirus, HSV, coronavirus, bovine papilloma virus, Mo-MLV, preferably adeno associated virus.

In another preferred embodiment, the vector of the present invention comprises a promoter, more preferably wherein said promoter is operably linked to the polynucleotides of the present invention.

In a preferred embodiment, said promoter is selected from, but not limited to, prokaryotic promoters, preferably wherein the prokaryotic promoter comprises further elements, such as a RNA polymerase binding site, for example a Pribnow box or parts thereof, such as a −35 element or parts thereof. Prokaryotic vectors of the invention can be used for recombinant expression of inactivated GtxA in e.g. E. coli. As E. coli does not contain the GtxC gene, the GtxA expressed in E. coli is not properly acylated and consequently non-toxic.

In another preferred embodiment, said promoter is selected from, but not limited to, eukaryotic promoters, preferably wherein the eukaryotic promoter comprises further elements, such as a RNA polymerase binding site, for example a TATA box or parts thereof, such as at least one binding site for any eukaryotic transcription factor.

A preferred embodiment of a vector of the invention is a naked DNA vaccine, comprising a eukaryotic promoter operatively linked to a polynucleotide of the invention.

### Vaccine

In general, a vaccine is a substance or composition capable of inducing an immune response in a living specimen with a functional immune system. The composition may comprise one or more of the following: an “active component” such as an antigen(s) (e.g. protein, polypeptides, peptides, nucleic acids and the like), nucleic acid constructs comprising one or more antigens amongst other elements, cells, (e.g. loaded APC, T cells for adoptive transfer), complex molecules (antibodies, TCRs and MHC complexes and more), carriers, adjuvants and pharmaceutical carriers. The present invention relates to a vaccine composition comprising an isolated polypeptide of the invention from Gallibacterium anatis, preferably an inactivated form of said polypeptide, or an immunogenic variant or fragment hereof. The term ‘vaccine’ used herein refers to a veterinary vaccine for the purpose of inducing a specific immunity against a disease originating from Gallibacterium anatis.

The present invention relates to a vaccine composition, comprising the polypeptide of SEQ ID No. 1, 2 or 3, an inactivated form of said polypeptides, a functional homologue thereof, a polypeptide with at least 70% sequence identity or an immunogenically active fragment of said polypeptides. Said vaccine is termed a toxoid vaccine. A toxoid vaccine is a vaccine wherein a toxin, which has lost its toxicity but retained its immunogenicity, is used to provoke an immune response in a target organism, said target organism becoming resistant to future infections with said toxins or similar toxins originating from identical or similar bacterial species.

In a preferred embodiment, said vaccine comprises inactivated polypeptides, said polypeptides being inactivated with respect to toxicity, but remaining immunogenic, are inactivated, preferably by heat, more preferably by exposure to a chemical such as formaldehyde, more preferably by expressing the polypeptide of SEQ ID No. 1 in a non-acylated form.

In another preferred embodiment, said vaccine further comprises inactivated or live, attenuated Gallibacterium anatis.

In yet another preferred embodiment, the present invention further relates to at least one other antigen from a virus or a microorganism pathogenic to an avian species, wherein said virus or microorganism is selected from, but not limited to, the group consisting of Infectious Bronchitis Virus, Newcastle Disease Virus, Infectious Bursal Disease Virus, Chicken Anaemia agent, Avian Reovirus, Avian Pneumovirus, Chicken Poxvirus, Avian Encephalomyelitis Virus, Mycoplasma gallisepticum, Haemophilus paragallinarum, Pasteurella multocida and Eschericia coli.

When an antigen, such as a toxin, is introduced into a host organism, additional components are usually used to boost the immune response. Such components are commonly referred to as adjuvants. The vaccine composition of the present invention preferably comprises an adjuvant and/or a carrier. Adjuvants are any substance whose admixture into the vaccine composition increases or otherwise modifies the immune response to the GtxA polypeptide or immunogenic fragments thereof. Carriers are scaffold structures, for example a polypeptide or a polysaccharide, to which the GtxA polypeptide or immunogenic fragment thereof is capable of being associated and which aids in the presentation the antigen.

Thus, in a preferred embodiment, the vaccine composition of the present invention comprises and adjuvant and/or carrier selected from, but not limited to, the group of Freund's complete and incomplete adjuvant, vitamin E, non-ionic block polymers, muramyldipeptides, quil A, mineral and non-mineral oil, vegetable oil and carbopol. The vaccine of the present invention may also comprise an emulsifier, such as Span or Tween.

A vaccine composition of the present invention may be administered by several routes, for example by intra muscular or subcutaneous injection, orally through e.g. food or water, such as by aerosols, for example by scarification e.g. in the foot or wing web, such as by eye drops, for example by in-ovo administration.

### Birds

Most members of the Pasteurellaceae family live as commensals in the mucosa of warm blooded animals, preferably birds. Members of the Gallibacterium genus include both commensal and pathogenic strains, wherein said pathogenic strains mainly cause disease in the respiratory and reproductive tracts of avian hosts.

In a preferred embodiment, the present invention relates to the polypeptide of the invention, the polynucleotide of the invention, and the vector of the invention for the treatment of a bacterial infection in a warm blooded animal, more preferably in an avian species, such as Anas, for example Anser, such as Aythya, for example Biziura, such as Branta, for example Cygnus such as Creagrus, for example Gelochelidon, such as Larus, for example Pagophila, such as Xemaes, for example Ciconiidae, such as Columba, for example Columbina, such as Ducula, for example Gallicolumba, such as Geopelia, for example Geotrygon, such as Goura, for example Gymnophaps, such as Hemiphaga, for example Leptotila, such as Leucosarcia, for example Macropygia, such as Metriopelia, for example Ocyphaps, such as Oena, for example Patagioenas, such as Phapitreron, for example Ptilinopus, such as Scardafella, for example Streptopelia, such as Treron, for example Turtur, such as Zenaida, for example Aepypodius, such as Alectura, for example Phasianidae, such as Tetraoninae, for example Pelecanidae, such as Phoenicopteridae, for example Cacatuidae, such as Loriidae, for example Psittacidae, such as Dromaiidae, for example Pterocnemia, such as Rhea for example Struthionidae.

In a more preferred embodiment, the polypeptide of the invention, the polynucleotide of the invention, and the vector of the invention is used in the treatment of a bacterial infection in an avian species is selected from the group consisting of ducks, turkeys and chickens, more preferably egg-laying hens.

### Antibodies

In order to detect the presence of the polypeptides of the invention or immunogenic fragments hereof, it is useful to generate antibodies capable of binding specifically to said polypeptides or immunogenic fragments hereof. Said antibodies may bind to any epitope on said polypeptides.

In a preferred embodiment, said antibodies may be serum-derived polyclonal antibodies or monoclonal or recombinant antibodies, wherein said antibodies comprising antigen binding fragments of antibodies such as Fv, scFv, Fab, Fab′ or F(ab)₂, multimeric forms such as dimeric IgA molecules or pentavalent IgM, affibodies or diabodies.

In a preferred embodiment, the present invention relates to an IgA antibody, most preferably a chicken IgA antibody.

Thus, in a preferred embodiment, the present invention relates to antibodies capable of binding specifically to a polypeptide of SEQ ID No. 1, 2 or 3.

In another preferred embodiment, the present invention relates to an antibody capable of binding to a polypeptide comprising a sequence identity of at least 70% to SEQ ID No. 1, more preferably 75% sequence identity, for example at least 80% sequence identity, such as at least 85% sequence identity, for example at least 90% sequence identity, such as at least 95% sequence identity, for example at least 96% sequence identity, such as at least 97% sequence identity, for example at least 98% sequence identity, such as at least 99% sequence identity to SEQ ID No. 1.

In yet a preferred embodiment the present invention relates to an antibody capable of binding to a polypeptide comprising a sequence identity of at least 70% to SEQ ID No. 2, more preferably 75% sequence identity, for example at least 80% sequence identity, such as at least 85% sequence identity, for example at least 90% sequence identity, such as at least 95% sequence identity, for example at least 96% sequence identity, such as at least 97% sequence identity, for example at least 98% sequence identity, such as at least 99% sequence identity to SEQ ID No. 2.

In another preferred embodiment the present invention relates to an antibody capable of binding to a polypeptide comprising a sequence identity of at least 70% to SEQ ID No. 3, more preferably 75% sequence identity, for example at least 80% sequence identity, such as at least 85% sequence identity, for example at least 90% sequence identity, such as at least 95% sequence identity, for example at least 96% sequence identity, such as at least 97% sequence identity, for example at least 98% sequence identity, such as at least 99% sequence identity to SEQ ID No. 3.

In another preferred embodiment the present invention relates to an antibody capable of binding to a polypeptide comprising an immunogenic fragment of any of the polypeptides of SEQ ID No. 1, 2 and 3, said fragments consists of at least 150 contiguous amino acids, preferably at least 200 amino acids more preferably at least 300 amino acids, more preferably at least 500 amino acids, more preferably at least 750 amino acids, more preferably at least 1000 amino acids, more preferably at least 1250 amino acids, more preferably at least 1500 amino acids, more preferably at least 1750 amino acids, more preferably at least 2000 amino acids.

In yet a preferred embodiment the present invention relates to an antibody capable of binding to a polypeptide comprising an immunogenic fragment, wherein said fragments may contain up to 30 amino acid substitutions, more preferably up to 25 substitutions, more preferably up to 20 substitutions, more preferably up to 15 substitutions, more preferably up to 10 substitutions, more preferably up to 5 substitutions such as at four, three, two or one substitutions.

### Diagnosis

A diagnostic test kit is a collection of all components for carrying out a method of diagnosis relating to the present invention.

In a preferred embodiment, the present invention relates to a test kit, wherein an indication of a bacterial infection resulting from the presence of a bacterial species from the Gallibacterium genus is detected in a biological sample. Said indication may be the presence of any polypeptides of SEQ ID No. 1, 2 or 3 or functional variants hereof or antibodies against any of said polypeptides.

In a preferred embodiment, the presence of said polypeptides or antibodies may be detected by an enzyme-linked immunosorbent assay (ELISA). ELISA is a quantitative technique used to detect the presence of protein, or any other antigen, in a sample. In ELISA an unknown amount of antigen is affixed to a surface, and then a specific antibody is washed over the surface so that it can bind to the antigen. This antibody is linked to an enzyme, and in the final step a substance is added that the enzyme can convert to some detectable signal.

### Several Types of ELISA Exist:

Indirect ELISA
Sandwich ELISA
Competitive ELISA
Reverse ELISA

Other immuno-based assays may also be used to detect said polypeptides or said antibodies in a sample, such as chemiluminescent immunometric assays and Dissociation-Enhanced Lanthinide Immunoassays.

The invention further relates to a diagnostic test kit for detecting the presence of any polynucleotides of the invention or other specific DNA or RNA sequences specific to Gallibacterium anatis GtxA.

Thus, in a preferred embodiment, the present invention relates to a polymerase chain reaction (PCR) or real time (RT)-PCR method to detect said polynucleotides. PCR is a technique to amplify, and thereby ultimately detect, a single or few copies of a piece of DNA across several orders of magnitude, generating thousands to millions of copies of a particular DNA sequence. The method relies on thermal cycling, consisting of cycles of repeated heating and cooling of the reaction for DNA melting and enzymatic replication of the DNA. Primers (short DNA fragments) containing sequences complementary to the target region along with a DNA polymerase are key components to enable selective and repeated amplification. As PCR progresses, the DNA generated is itself used as a template for replication, setting in motion a chain reaction in which the DNA template is exponentially amplified.

### Medical Use of Polypeptides, Polynucleotides and Expression Vectors

Inactivated toxins as toxoid vaccines are commonly used in the treatment and/or prophylactic treatment of bacterial infections, predominantly for veterinarian application, preferably for treating poultry flocks.

With a toxoid vaccine, the goal is to condition the immune system to combat not an invading bacterium but rather a toxin produced by that invading bacterium.

Therefore, the GtxA polypeptide, polynucleotides encoding the GtxA polypeptide or expression vectors of the invention may be used to generate a toxoid vaccine for the treatment and/or prophylactic treatment of pathogenic conditions caused by bacteria secreting the GtxA toxin and/or similar toxins.

In a particular embodiment the dosage for providing passive immunity to birds is about 0.25 ml per dose of the vaccine. In another embodiment the dosage is about 0.4 ml per dose of the vaccine. In still another embodiment the dosage is about 0.6 ml per dose of the vaccine. In an embodiment, the dosage is about 0.5 ml per dose of the vaccine. In one embodiment the avian is selected from, but not limited to, the group consisting of ducks, turkeys and chickens. In another preferred embodiment the avian is an egg-laying hen.

The present invention also provides a method of administering a vaccine of the present invention to an avian to protect it against multiple diseases by including other vaccines into the composition.

The present invention includes for the vaccination of birds, preferably ducks, turkeys or chickens, more preferably egg-laying hens, to provide active immunity against GtxA toxin. The chicks and/or poults are vaccinated early in life, at about day one of age or slightly older (within the first week of life) with a single or two doses of the vaccine. Appropriate vaccine dosages for achieving active immunity can vary from about 0.05 ml to about 0.5 ml. In a particular embodiment the vaccine dosage is about 0.05 ml to about 0.1 ml.

It is expected that each bird needs to be vaccinated more than once, such as for example two time within 1-3 months. Yearly revaccination may be required for life-long protection of e.g. egg-laying hens.

In yet further embodiments, a toxin or immunogenic fragment thereof of this invention can be administered into the bird. Also, the dosage range of a GtxA toxin or immunogenic fragment thereof used as a vaccine of this invention can be from about 1 μg/kg bodyweight to about 1000 μg/kg bodyweight per dose, with an exemplary range of about 10 μg/kg bodyweight to about 100 μg/kg bodyweight per dose per animal.

TABLE I
Primer list. Primers used for construction and
verification of gtxA mutants. Restriction sites
are underlined.

| Primer | Primer sequence | |
|---|---|---|
| Name | (5′-3′) | SEQ ID NO |
| 4240 | TATCGTCGACTATCCATCGCG | 7 |
| | GCATCAG | |
| 4242 | AGCTGAATTCAAGCAAGTGCT | 8 |
| | ATTGCTACCG | |
| 4243 | AGCTGAATTCTTATGTCGGCG | 9 |
| | ATCAAACAA | |
| 4245 | TATGTCTAGAGGCGTTGGTG | 10 |
| | GATAAGAGAT | |
| kanR | CGATAGATTGTCGCACCTGA | 11 |
| kanF | TATGGAACTGCCTCGGTGA | 12 |
| 39F | TGATGCAATCAAAGATAAAGT | 13 |
| | CG | |
| 5734R | AATCGGCATTGGAGCTTTC | 14 |
| 2871F | AACCAAACCAATCCAAGGT | 15 |
| 3270R | ATTGCCGTCTTTGCCTACTG | 16 |

TABLE II
List of primers used for constructs for expression in E. coli.
Restriction sites are underlined. Bold indicates overlapping
regions for splicing by overlap extension.

| | Primer sequence | | | |
|---|---|---|---|---|
| Primer name | (5′-3′) | Construct | Primers used | SEQ ID NO |
| GtxUP-NcoI | AGTCCCATGGGT | gtxA + C | GtxUP-NcoI & | 17 |
| | CTTTCATTAAAAG | | gtxC-r-XhoI | |
| | AAAAAGTAACTG | | | |
| | GAATA | | | |
| gtxC-r-XhoI | CAGTCTCGAGTT | gtxA | GtxUP-NcoI & | 18 |
| | ATGAATTTTCTTC | | gtxA-r-XhoI | |
| | TATAAAAGCAGC | | | |
| gtxA Cf NcoI | AGTCCCATGGCA | RTX + C | gtxA cf NcoI & | 19 |
| | ATTGAATCTTTCA | | gtxC-r-XhoI | |
| | ATTTAATCGCAA | | | |
| gtxA-Nr-XhoI | CAGTCTCGAGTT | RTX | gtxA cf NcoI & | 20 |
| | AATTTAGGAAATC | | gtxA-r-XhoI | |
| | GGTCATTATGCC | | | |
| | AT | | | |
| gtxA-r-XhoI | CAGTCTCGAGTT | Nterm + C | GtxUP-NcoI & | 21 |
| | AAACAAGATACAT | | SOErev1 | |
| | AGTGACCAGTTC | | | |
| | AT | | | |
| SOErev1 | GTTATCCATAAT | | SOEfor2 & gtxC-r- | 22 |
| | AATTAATTTAGGA | | XhoI | |
| | AATCGGTCATTAT | | | |
| | G | | | |
| SOEfor2 | TTCCTAAATTAAT | Nterm | GtxUP-NcoI & | 23 |
| | TATGGATAACTTC | | gtxA-r-XhoI | |
| | TCAACTTTAGG | | | |

### EXAMPLES

### Example 1

### GtxA from Gallibacterium anatis, a Cytolytic RTX-Toxin with a Novel Domain Organisation

### Abstract

Gallibacterium anatis is a pathogen in chickens and other avian species where it is a significant cause of salpingitis and peritonitis. We found that bacterial cells and cell-free, filter-sterilised culture supernatant from the haemolytic G. anatis biovar haemolytica were highly cytotoxic towards avian-derived macrophage-like cells (HD11). We obtained the genome sequence of G. anatis 12656-12 and used a rational approach to identify a gene predicted to encode a 2026 amino acid RTX-toxin, which we named GtxA (Gallibacterium toxin). The construction of a gtxA knock-out mutant showed gtxA to be responsible for G. anatis' haemolytic and leukotoxic activity. In addition, E. coli expressing gtxA and an adjacent acyltransferase, gtxC, became cytolytic. GtxA was expressed during in vitro growth and was localised in the extracellular protein fraction in a growth phase dependent manner. GtxA had an unusual modular structure; the C-terminal 1000 amino acids of GtxA were homologous to the classical pore-forming RTX-toxins in other members of Pasteurellaceae. In contrast, the N-terminal approximately 950 amino acids had few significant matches in sequence databases. Expression of truncated GtxA proteins demonstrated that the C-terminal RTX-domain had a lower haemolytic activity than the full-length toxin, indicating that the N-terminal domain was required for maximal haemolytic activity. Cytotoxicity towards HD11 cells was not detected with the C-terminal alone, suggesting that the N-terminal domain plays a critical role for the leukotoxicity.

### Materials and Methods

### Bacterial Strains and Growth Conditions

Gallibacterium anatis biovar haemolytica strain 12656-12 Liver (referred to as 12656-12) was used in this study, this strain was originally isolated from the liver of a septicaemic chicken [Bojesen A. M., Torpdahl M., Christensen H., Olsen J. E., Bisgaard M., Genetic diversity of Gallibacterium anatis isolates from different chicken flocks, J. Clin. Microbiol. (2003) 41:2737-2740]. G. anatis 12656-12 was grown at 37° C. either on brain heart infusion (BHI) (Oxoid) agar supplemented with 5% citrated bovine blood in a closed plastic bag, or in BHI broth with aeration. Anaerogen (Oxoid) was used to produce anaerobic conditions in incubator jars. E. coli strains were grown in Luria-Bertani broth and agar, the medium was supplemented with 50 μg/mL kanamycin and 20 μg/mL chloramphenicol when appropriate. All chemicals were purchased from Sigma.

### Bioinformatics Analyses

A draft version (115 contigs) of the genome sequence of G. anatis biovar haemolytica 12656-12 Liver (A. M. Bojesen, unpublished data) was obtained from 454 Life Sciences, using the pyrosequencing-based method [Margulies M., Egholm M., Altman W. E., Attiya S., Bader J. S., Bemben L. A., et al., Genome sequencing in microfabricated high-density picolitre reactors, Nature (2005) 437:376-380]. Gene annotation was performed using Wasabi, a web-based annotation system for prokaryotic organisms provided by the Victorian Bioinfomatics Consortium, Monash University, Australia [Bulach D. M., Zuerner R. L., Wilson P., Seemann T., McGrath A., Cullen P. A., Davis J., Johnson M., Kuczek E., Alt D. P., Peterson-Burch B., Coppel R. L., Rood J. I., Davies J. K., Adler B., Genome reduction in Leptospira borgpetersenii reflects limited transmission potential, Proc. Natl. Acad. Sci. USA (2006) 103:14560-14565]. Sequence similarity searches were performed using BLASTP [Altschul S. F., Madden T. L., Schaffer A. A., Zhang J. H., Zhang Z., Miller W., Lipman D. J., Gapped BLAST and PSI-BLAST: a new generation of protein database search programs, Nucleic Acids Res. (1997) 25:3389-3402] (database: non-redundant protein sequences (GenBank) and SwissProt), FASTA [Pearson W. R., Lipman D. J., Improved tools for biological sequence comparison, Proc. Natl. Acad. Sci. USA (1988) 85:2444-2448] and SSEARCH (databases: UniProtKB and SwissProt), and HHpred [Soding J., Biegert A., Lupas A. N., The HHpred interactive server for protein homology detection and structure prediction, Nucleic Acids Res. (2005) 33:W244-W248] (database: Interpro (2009)). All searches were performed in March 2009. Transterm [Kingsford C. L., Ayanbule K., Salzberg S. L., Rapid, accurate, computational discovery of Rho-independent transcription terminators illuminates their relationship to DNA uptake, Genome Biol. (2007) 8:R22] was used to predict transcriptional terminators.

### Haemolysis Assay

The haemolytic activity was assayed as previously described [Rowe G. E., Welch R. A., Assays of Hemolytic Toxins, Methods Enzymol. (1994) 235:657-667]; bovine blood was washed repeatedly in TN Buffer (10 mM Tris-HCl, 0.9% NaCl, pH 7.5) until the upper phase appeared colourless. A 2% (vol/vol) erythrocyte solution was prepared in TN-buffer supplemented with 10 mM CaCl₂. Erythrocytes were incubated with filter-sterilised bacterial culture supernatant or bacteria in a 1:1 ratio at 37° C. for one hour unless otherwise noted. Un-lysed erythrocytes and cell debris were collected by centrifugation and the amount of released haemoglobin was measured in a plate reader at 540 nm. 100% lysis was determined with 1% triton-X and background lysis was subtracted before calculation of haemolytic activity. The effect of heat was examined by incubating the supernatant at 60° C. for 30 min before the haemolysis assay. The effect of proteinase K was examined by incubating the supernatant with 4 μg/mL proteinase K at 37° C. for 30 min before haemolysis assay.

### Culturing of HD11 Cells and LDH Cytotoxicity Assay

The macrophage-like cell line HD11 derived from MC29 transformation of chicken bone marrow cells [Beug H., Vonkirchbach A., Doderlein G., Conscience J. F., Graf T., Chicken Hematopoietic-Cells Transformed by 7 Strains of Defective Avian Leukemia Viruses Display 3 Distinct Phenotypes of Differentiation, Cell (1979) 18:375-390] was maintained in Roswell Park Memorial Institute (RPMI) 1640 medium+GlutaMAX™-I+25 mM HEPES (Gibco). The media was supplemented with 2.5% chicken serum, 7.5% foetal bovine serum (FBS), and 25 μg/mL gentamicin. The cells were cultured as an adherent cell line at 37° C. with an atmosphere of 5% CO₂ and were sub-cultured every 2nd or 3rd day. For the cytotoxicity assays the cells were seeded in 96 well plates with 2×10⁴ cells in RPMI added 5% FBS in a total volume of 100 μL. The cells were incubated overnight, and the media was changed. Filter-sterilised culture supernatant or bacteria resuspended in saline (0.9% NaCl) was added to the cells and incubated for one hour. For E. coli, expression of recombinant proteins were induced as described in section 2.7 and the OD₆₀₀ (optical density at 600 nm) was adjusted to 1 corresponding to approx. 6×10⁸ CFU/mL. G. anatis cells and supernatant was harvested in late exponential phase (OD₆₀₀=1). The suspension of G. anatis was adjusted to OD₆₀₀ of 1 corresponding to approx. 4×10⁸ CFU/mL. Filter-sterilised culture supernatant was stored on ice and added to cells within 30 minutes after harvest. Cytotoxicity was determined with LDH cytotoxicity assay (Promega) as described by the manufacturer. Each sample was assayed in triplicate wells and the experiments were repeated a minimum of three times.

Construction of a G. anatis gtxA mutant

A 1508 bp fragment consisting of nucleotides 140 to 1648 of gtxA was PCR-amplified with primers 4240 and 4242, and a 1483 bp fragment consisting of nucleotides 3995 to 5478 was amplified with primers 4243 and 4245 (primer sequences are listed in Tab. I). The two fragments were digested with restriction enzymes and ligated into the corresponding restriction sites in plasmid pWSK129 [Wang R. F., Kushner S. R., Construction of versatile low-copy-number vectors for cloning, sequencing and gene expression in Escherichia coli, Gene (1991) 100:195-199]. The gel-purified kanamycin-cassette (Tn903) from EcoRI-digested pUC4-KISS [Barany F., 2-Codon Insertion Mutagenesis of Plasmid Genes by Using Single-Stranded Hexameric Oligonucleotides, Proc. Natl. Acad. Sci. USA (1985) 82:4202-4206] was ligated into the EcoRI site between the two PCR-fragments. The kanamycin resistance gene was inserted in the same transcriptional direction as gtxA. The plasmid DNA was linearised by digestion with XhoI and SalI and column purified. The natural competence of G. anatis 12656-12 was induced by the MIV-method as previously described for Haemophilus influenzae [Poje G., Redfield R. J., Transformation of Haemophilus influenzae, Methods Mol. Med. (2003) 71:57-70]; G. anatis was grown in BHI to an OD₆₀₀ of 0.2, washed once in MIV and incubated in MIV for 100 min. The linear DNA was added to the cells at a concentration of 0.5 μg DNA/mL. After 20 min, two volumes of BHI were added and the bacteria were incubated for 1 h before transformants were selected on blood agar plates with 5 μg/mL kanamycin. Colonies were re-streaked and the deletion was verified with primer pairs 39F+kanR, kanF+5334R and 2871F+3270R. The strain was designated ΔgtxA.

### Construction of Expression Plasmids

Plasmids encoding full length GtxA, the N-terminal domain of GtxA (amino acids 1-949) and the RTX-domain of GtxA (amino acids 931-2026) with and without GtxC were constructed by the ligation of PCR fragments into the expression vector pET28a (Novagen). The PCR fragments were amplified with pfx50 polymerase (Invitrogen), column purified, double digested with NcoI and XhoI and either column purified again or gel-purified (fragments >6 kb). The primers used for each construct are listed in Table II. The construct Nterm+C, nucleotides 1-2847 of gtxA in operon with gtxC, was made by the use of splicing by overlap extension [Horton R. M., Cai Z. L., Ho S, N., Pease L. R., Gene-Splicing by Overlap Extension—Tailor-Made Genes Using the Polymerase Chain-Reaction, Biotechniques (1990) 8:528-535], where the primers GtxUP-NcoI and gtxC-r-XhoI were used in the second round of PCR. Plasmid pET28a was double digested with NcoI and XhoI, dephosphorylated with Antarctic phosphatase (NEB) and gel purified. Vector and PCR-fragments were ligated at a molar ratio of 1:3, transformed into chemically competent E. coli Top10F″ (Invitrogen) and selected on LB-agar plates with kanamycin. The sequence of the insert in each plasmid was verified by DNA-sequencing (Macrogen, Korea). The plasmids were transformed into the E. coli expression strain ER2566 (New England Biolabs). Plasmid pLG575 encodes E. coli hIyB and h/yD, components of the T1SS secreting HIyA [Mackman N., Nicaud J. M., Gray L., Holland I. B., Genetical and functional organisation of the Escherichia coli haemolysin determinant 2001, Mol. Gen. Genet. (1985) 201:282-288], and was introduced to promote secretion of the expressed proteins.

Expression of Recombinant GtxA Proteins in E. coli

Protein expression was induced on agar plates containing 0.1 mM IPTG incubated at 30° C. For induction in broth, an overnight culture was diluted 1:50 and incubated at 37° C. with shaking until the culture reached an OD₆₀₀ of 0.6. Then, IPTG (0.2 mM) was added and induction was maintained for two hours at 30° C. To release recombinant protein from the cells, cells were pelleted by centrifugation, resuspended in 0.1 M Tris/0.9% NaCl in 1/25 of the original volume, lysed by bead beating (FastPrep) for 45 seconds and spun down at 4° C., and the supernatant was used immediately for liquid haemolysis assays and LDH cytotoxicity assay.

### SDS Page and Western Blot Analysis

Total cellular protein was obtained by harvesting 500 μL of culture and resuspending the cell pellets in 10 mM Tris, 500 μL/per OD unit at the time of harvest. Extracellular proteins were prepared from filter-sterilised culture supernatant (low protein binding filter (0.22 μm) (Millex® GP (Millipore)). Proteins were precipitated overnight by the addition of one volume ice-cold 96% ethanol, collected by centrifugation (13000 g for 30 min. at 0° C.), and resuspended in 10 mM Tris (1/100 of the original volume). Proteins were separated by SDS-PAGE in NuPAGE® Novex gels (Invitrogen). For Western blot analysis, proteins were transferred to polyvinylidene difluoride membranes (Invitrogen). The primary antibody, rabbit antiserum raised against ApxI from A. pleuropneumoniae [Schaller A., Kuhn R., Kuhnert P., Nicolet J., Anderson T. J., Maclnnes J. I., Seger R. P. A. M., Frey J., Characterization of apxIVA, a new RTX determinant of Actinobacillus pleuropneumoniae, Microbiology (1999) 145:2105-2116], was used at a 1:1333 dilution and detected with Westernbreeze Chemiluminiscent Western Blot Immunodetection Kit (Invitrogen) as described by the manufacturer.

### RNA Purification

An overnight culture was diluted 1:100 in BHI and incubated at 37° C. with aeration. Cells were harvested at OD₆₀₀ 0.17, 0.6, 2, 3, as well as one hour after growth had stopped and after 24 hours of incubation. Total RNA was isolated with RNeasy protect Mini Kit (Qiagen), on-column DNAse treatment was performed as described by the manufacturer (Qiagen).

### Northern Blotting

Blotting of RNA, probe labelling (with [α-³²P]-dCTP) and hybridization was performed basically as described [Frees D., Chastanet A., Qazi S., Sorensen K., Hill P., Msadek T., Ingmer H., Clp ATPases are required for stress tolerance, intracellular replication and biofilm formation in Staphylococcus aureus, Mol. Microbiol. (2004) 54:1445-1462]. A 384 bp fragment within the RTX-half of gtxA was PCR-amplified with primers 3487F 5′-GCCTCTACCGCCGTTTCTG-3′ and 3874R 5′-GGCTGGCTAATAATTCATCACCTTG-3′ and used as template in the probe labelling reaction.

### Results

Cytolytic Activity of G. anatis

G. anatis biovar haemolytica is β-haemolytic on bovine-blood agar plates [Christensen H., Bisgaard M., Bojesen A. M., Mutters R., Olsen J. E., Genetic relationships among avian isolates classified as Pasteurella haemolytica, ‘Actinobacillus salpingitidis’ or Pasteurella anatis with proposal of Gallibacterium anatis gen. nov., comb. nov and description of additional genomospecies within Gallibacterium gen. nov, Int. J. Syst. Evol. Microbiol. (2003) 53:275-287]. To test the target range we examined the haemolysis of strain 12656-12 on agar plates with blood from different species, and the bacterium produced clear zones of β-haemolysis when grown on agar plates containing horse, cow, swine or chicken blood (data not shown), confirming the broad target range reported by Greenham and Hill [Greenham L. W., Hill T. J., Observations on an Avian Strain of Pasteurella haemolytica, Vet. Rec. (1962) 74:861-863]. The bacterium was haemolytic under both aerobic and anaerobic culture conditions.

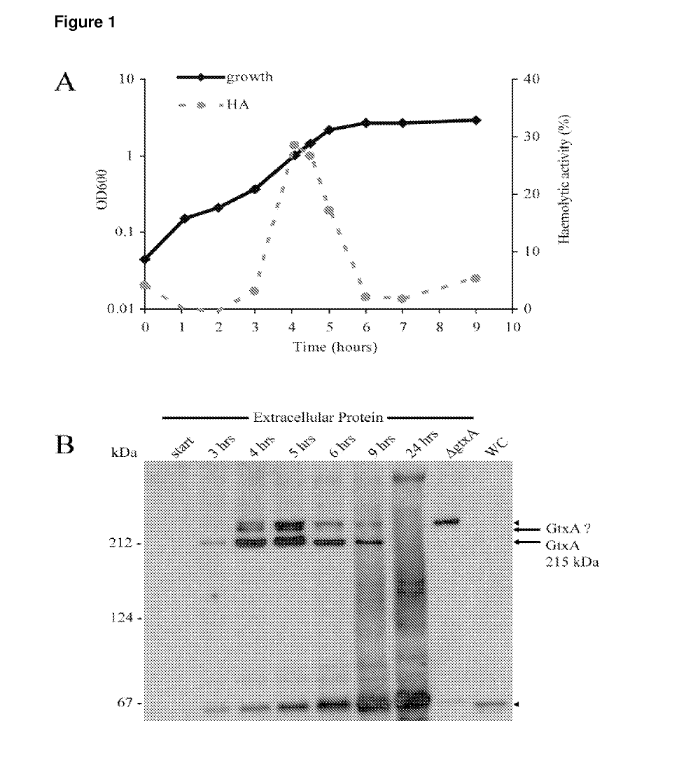

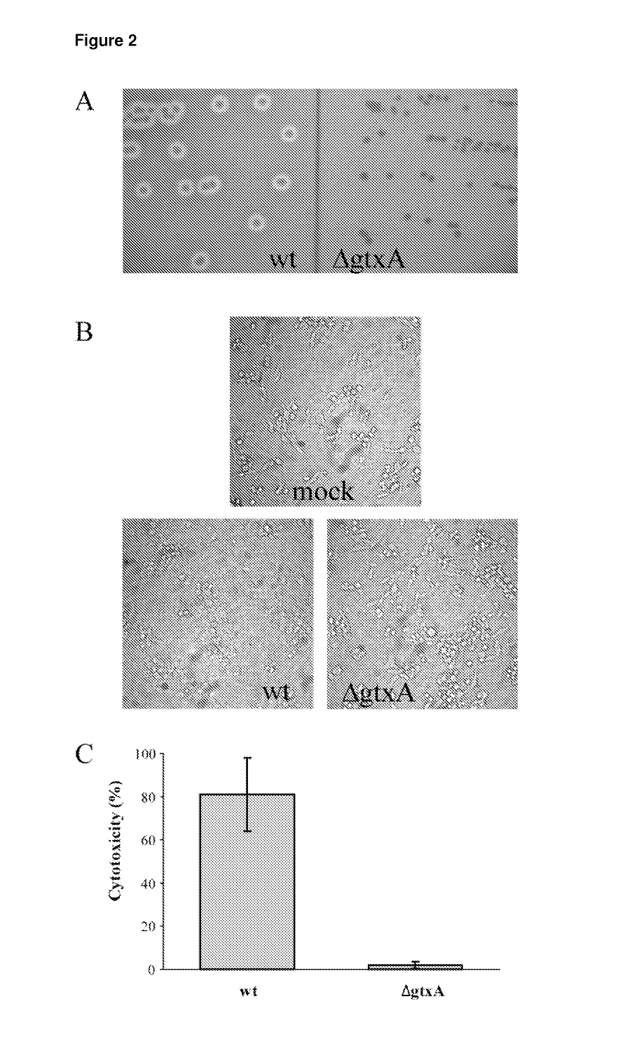

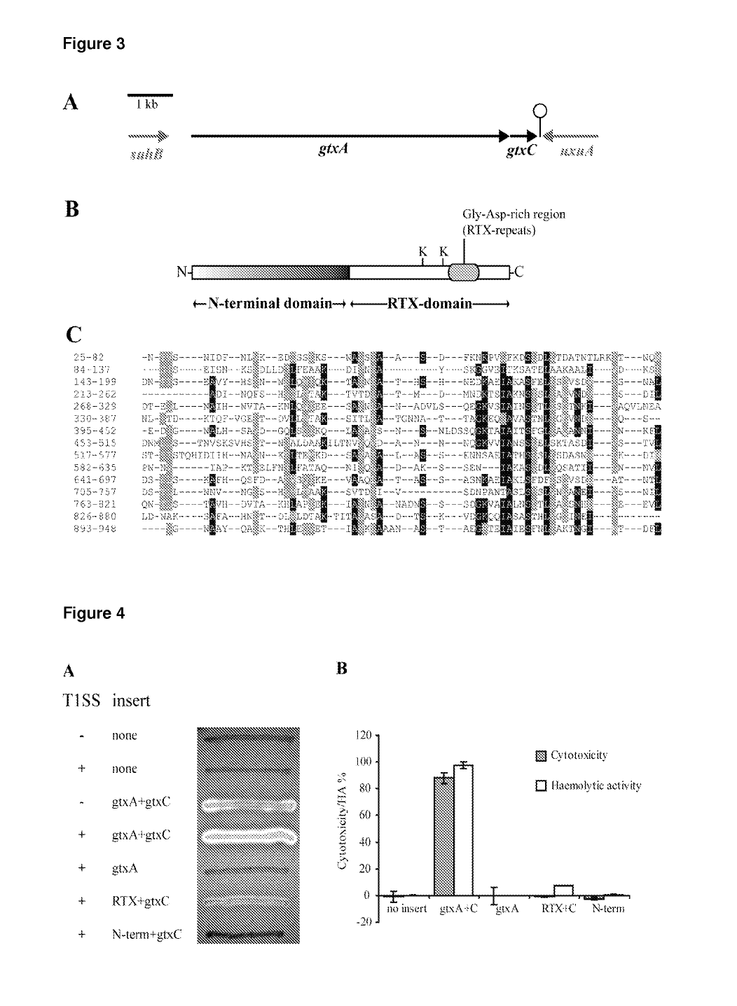

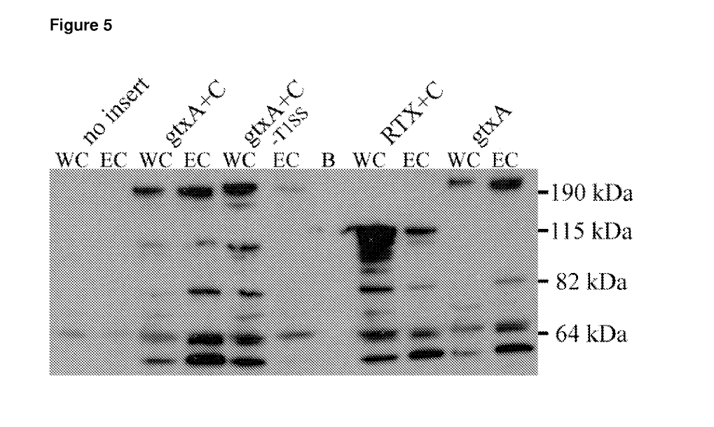

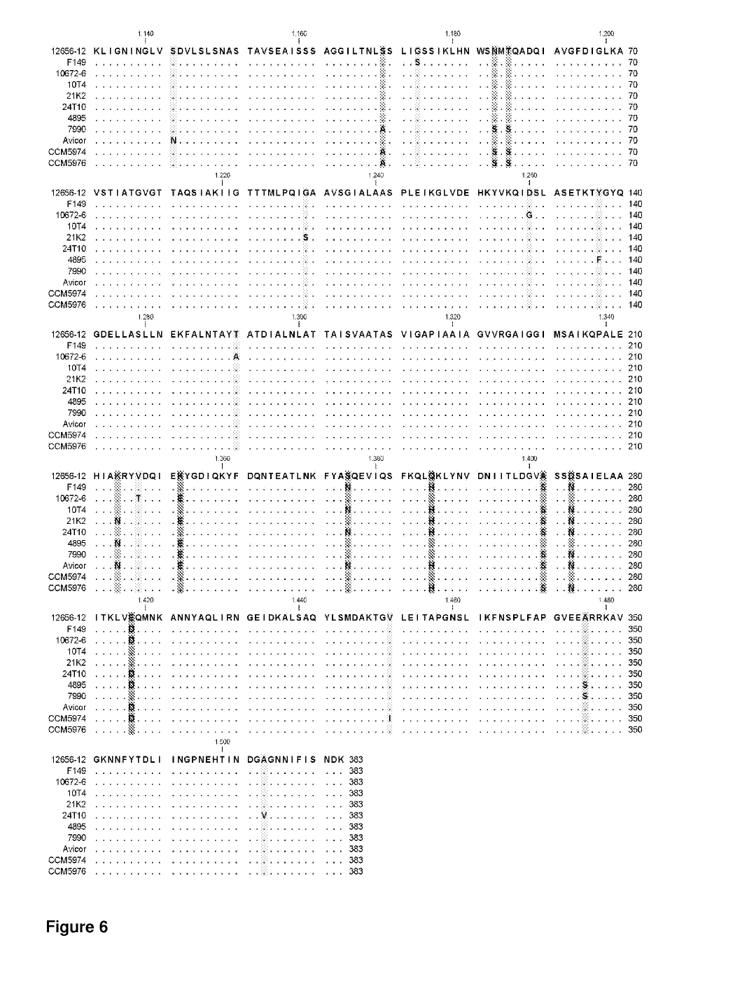

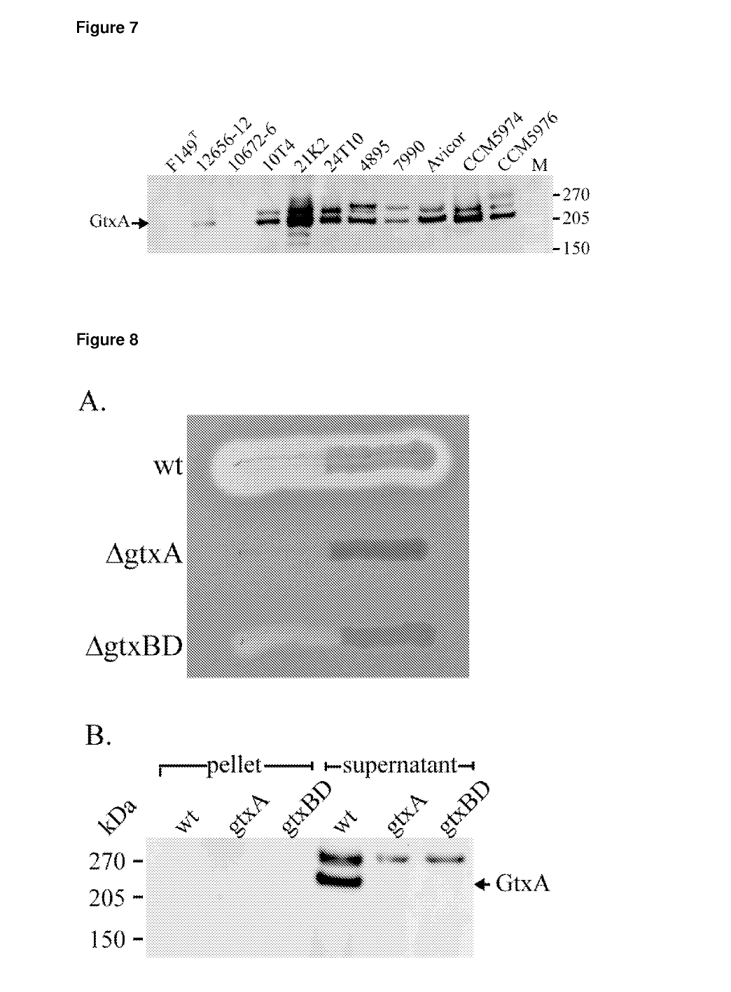

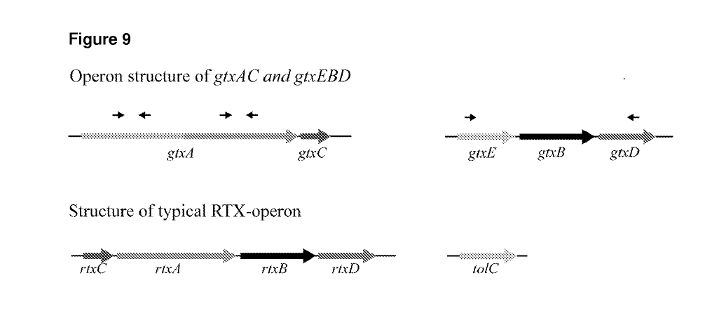

This haemolytic activity has been suggested to originate from a secreted haemolysin [Greenham L. W., Hill T. J., Observations on an Avian Strain of Pasteurella haemolytica, Vet. Rec. (1962) 74:861-863]. To test this, liquid haemolysis assays were performed, where G. anatis 12656-12 cell-free culture supernatants, harvested in different phases of growth, were incubated with a suspension of bovine erythrocytes and the amount of released haemoglobin measured. G. anatis supernatant from mid to late exponential phase lysed the erythrocytes efficiently (FIG. 1A). This activity was inactivated by heat (60° C.) and proteinase K, and was reduced by the calcium chelater EGTA (data not shown), confirming that G. anatis produces a calcium-dependent secreted haemolytic protein.

The lysis of erythrocytes may play a role in iron acquisition in the host, however, interactions with other types of cell, e.g. leukocytes, may play a more important role during natural infection. We therefore tested G. anatis' cytotoxic activity towards leukocytes using the avian-derived macrophage-like cell line HD11. The HD11 cells showed rounding and detached from the surface after exposure to G. anatis (FIG. 2B).

The cytotoxicity was quantified using the lactate dehydrogenase (LDH) cytotoxicity assay, which showed a pronounced cell death (FIG. 2C). This leukotoxic activity of G. anatis is likely to be essential in the pathogenesis of this bacterium and proteins responsible for the leukotoxic activity are thus expected to be important virulence factors.

Identification of an RTX-Toxin in G. anatis' Genome Sequence

To identify a specific protein responsible for G. anatis' cytotoxic phenotype, we obtained the genome sequence of G. anatis 12656-12 and searched for sequences encoding possible toxins. Proteins belonging the group of pore-forming RTX-toxins are important virulence factors and responsible for haemolytic and leukotoxic activity in bacteria related to Gallibacterium [Frey J., Kuhnert P., RTX toxins in Pasteurellaceae, Int. J. Med. Microbiol. (2002) 292:149-158], making proteins of this type an obvious target to search for. BLAST searches with the amino acid sequences of different RTX-toxins (including ApxI and ApxII from A. pleuropneumoniae, LtxA from A. aggregatibacter and HIyA from E. coli) against the G. anatis 12656-12 genome sequence led to the identification of a putative G. anatis RTX-toxin of 2026 amino acids. The 6081 nucleotide (nt) open reading frame (ORF) encoding this protein was named gtxA: gtx for Gallibacterium toxin and A by analogy to the designation of toxin gene in other RTX operons [Frey J., Kuhnert P., RTX toxins in Pasteurellaceae, Int. J. Med. Microbiol. (2002) 292:149-158]. gtxA is followed by a very short five nucleotides (nt) intergenic region and a 492 nt ORF (FIG. 3A) encoding a predicted protein of 163 amino acids. This protein has homology to acyltransferase proteins, which are required for activation of RTX-toxins, and showed 38% identity and 60% similarity to the acyltransferease HIyC from E. coli. By analogy, the gene was named gtxC. A rho-independent transcriptional terminator was found downstream of gtxC and probably marks the end of a transcriptional unit including both gtxA and gtxC. The genes flanking the gtxA-C operon were predicted to encode an inositol-1-monophosphatase (suhB upstream of gtxA), and a mannoate dehydratase gene (uxuA downstream of gtxC) (FIG. 3A), both of which are unlikely to be involved in GtxAC function. Interestingly, GtxA (2026 aa) is twice as large as the “typical” RTX-toxins (approx. 1000 aa [Frey J., Kuhnert P., RTX toxins in Pasteurellaceae, Int. J. Med. Microbiol. (2002) 292:149-158]) described from other members of the Pasteurellaceae family and HIyA from E. coli. The 1000 amino acids at the C-terminus of GtxA are homologous to these RTX-toxins, with whom the region shares approx. 20% sequence identity and 35% sequence similarity. This C-terminal region also contains several of the conserved features of RTX-toxins. HIyA from E. coli is acylated at Lys564 and Lys690 [Stanley P., Packman L. C., Koronakis V., Hughes C., Fatty Acylation of 2 Internal Lysine Residues Required for the Toxic Activity of Escherichia coli Hemolysin, Science (1994) 266:1992-1996]; both these lysine and the preceding glycine residues are conserved in GtxA (Lys1484 and Lys1607 (FIG. 3B)), so these are likely acylation sites in GtxA mediated by GtxC. Downstream of the predicted acylation sites (aa 1640-1830), GtxA has a glycine and aspartate-rich region, which is also a conserved feature of the RTX-toxins. In contrast, the N-terminal region (aa 1 to approx. 950) had limited similarity to available sequences, and no significant homologues were found by BLASTP searches against the GenBank database. However, the region from aa 520 to 879 had similarity (E-value 0.007) to a conserved domain (COG1511) of unknown function from predicted membrane proteins. Compared to the RTX-domain, the N-terminal domain is less acidic and contains a larger proportion of hydrophobic amino acids, particularly serine. The secondary structure was predicted to consist primarily of alpha-helices.

To get an idea of the function of the N-terminal domain, we performed a bioinformatic analysis of its amino acid sequence by the use of more sensitive search tools for sequence similarity and homology prediction. Homology searches using FASTA and SSearch found sequence similarity to the eukaryotic cytoskeletal proteins Talin-A and Talin-B from the amoeba Dictyostelium discoide (E-value 3.4×10⁻⁷ and 0.0061, respectively), and Talin from chickens (Gallus gallus) (E-value 0.0087). Furthermore, the homology detection server HHpred predicted homology to talin (probability=100%). Talin binds to a range of other proteins, including actin, vinculin and the cytosolic part of integrins. Large proteins often consists of repeats arisen by duplications and examination of the N-terminal domain with the repeat finder Radar [16], found 15 repeats of 57 amino acids (FIG. 3C).

GtxA thus consists of two domains: an N-terminal repeat domain and a C-terminal RTX/cytolysin domain.

GtxA has Cytolytic Activity which is Dependent on GtxC

To examine if GtxA is a cytolytic protein, gtxA was cloned together with the predicted acyltransferase gene, gtxC, and introduced into the non-haemolytic expression strain E. coli ER2566. Upon expression of gtxA and gtxC, this strain exhibited a haemolytic phenotype on blood agar plates and in liquid haemolytic assays (FIG. 4), showing that GtxAC holds haemolytic activity. RTX-toxins are usually extracellular proteins exported by specific T1SS. Introduction of a plasmid (pLG575) expressing the T1SS encoded by E. coli hIyBD increased the haemolysis zone (FIG. 4A) and immunoblotting showed that a larger fraction of GtxA was present in the extracellular protein fraction (FIG. 5), demonstrating that the E. coli secretion system can secrete G. anatis GtxA. The cytotoxic activity of GtxA towards HD11 cells was assayed by LDH release assay and E. coli ER2566 expressing gtxAC was toxic to HD11 cells. E. coli containing vector with no insert (negative control) showed no toxicity after one hour incubation (FIG. 4B). The requirement of post translational acylation is one of the hallmarks of RTX-toxins. To access whether this was also the case for the atypical GtxA, we examined the activity of GtxA expressed without its predicted acyltransferase GtxC. FIG. 4 shows that when GtxA was expressed in the absence of GtxC it had no cytolytic activity against erythrocytes or leukocytes. Thus, the non-acylated protoxin is inactive, and posttranslational acylation is essential for both its haemolytic and leukotoxic activities. The secretion of GtxA was not hindered by the lack of acylation, as the non-acylated GtxA was detected in the culture supernatant in amounts similar to the acylated toxin (FIG. 5).

GtxA is Responsible for G. anatis' Cytotoxic Activity

To determine whether G. anatis' haemolytic and leukotoxic activity originated from GtxA, we constructed a gtxA knock out mutant. No molecular tools for genetic manipulation of Gallibacterium had previously been described, but, we found that G. anatis 12656-12 is naturally competent, a trait we exploited in the construction of stable gtxA mutants by natural transformation. In the resulting mutants, the 2347 nucleotides between positions 1648 and 3995 in gtxA were deleted and replaced by a kanamycin resistance cassette.

In contrast to the wild-type, the gtxA mutant was not haemolytic on blood agar plates (FIG. 2A) or in liquid haemolysis assay (data not shown). Furthermore, gtxA showed no cytotoxicity towards HD11 cells (FIGS. 2B and 2C). Identical results were obtained from two independently constructed gtxA mutants. Thus, gtxA is responsible for the haemolytic and leukotoxic activity of G. anatis.

### Growth Phase Dependent Levels of GtXA and its Activity

The haemolytic activity of G. anatis supernatant was growth phase dependent: the activity peaked in late exponential phase, dropped at the transition to stationary phase, and was low in the supernatant from overnight cultures (FIG. 1A). This prompted us to hypothesise that the expression of GtxA was similarly growth phase dependent. To examine this and to establish GtxA's localisation, we determined the amount of GtxA in the culture supernatant (extracellular proteins) and whole cell lysates at different times throughout growth using immunoblotting with ApxI-antiserum (FIG. 1B). The ApxI-antiserum recognised several proteins in the extracellular protein fraction including a band corresponding to the size of the predicted molecular mass of full length GtxA (215 kDa). This band was absent in ΔgtxA, supporting that the band is GtxA. Like the haemolytic activity, the presence and amounts of GtxA in the supernatant were growth phase-dependent (FIG. 1B): the amount of GtxA peaked at the transition to stationary phase, and the protein was not detected in day-old cultures (24 hrs) which is similar to the pattern reported for A. pleuropneumoniae ApxI and ApxII [Jarma E., Regassa L. B., Growth phase mediated regulation of the Actinobacillus pleuropneumoniae ApxI and ApxII toxins, Microb. Pathog. (2004) 36:197-203] and M. haemolytica LktA [Strathdee C. A., Lo R. Y., Regulation of expression of the Pasteurella haemolytica leukotoxin determinant, J. Bacteriol. (1989) 171:5955-5962]. A second band (>215 kDa) was also present in wild-type but absent in gtxA suggesting that GtxA may exist in two different forms, possibly due to post translational modifications. Two further bands (65 kDa and >215 kDa, respectively) were detected in both wild-type and mutant and are likely not related to GtxA. No protein of the size of GtxA was detected in whole cell lysates at any time point, consistent with the predicted extracellular localisation and in support of GtxA being secreted immediately after or in connection with synthesis. To examine transcription of gtxA, northern blotting was performed with RNA from cells harvested throughout the growth phase. The blots showed gtxA to be transcribed during exponential growth and in the beginning of stationary phase, but no transcript was detected two hours into stationary phase and in overnight cultures, indicating the transcription of gtxA was shut down during stationary phase. In conclusion, GtxA is expressed during in vitro growth, it is a growth phase dependent extracellular protein and the growth phase dependence is influenced by transcriptional regulation, and the balance between accumulation of secreted GtxA and its subsequent degradation.

### The N-Terminal Domain of GtxA is Required for Full Cytolytic Activity

The bioinformatical analysis showed that GtxA has an atypical organisation compared to other pore-forming RTX-toxins consisting of two parts, an RTX-domain and an N-terminal domain (FIG. 3B). To examine the contribution of the N-terminal domain to the cytolytic activity of GtxA, both the N-terminal domain (amino acids 1-949) and the RTX-domain (amino acids 931-2026) were expressed separately in E. coli and their haemolytic and leukotoxic activities examined and compared to those of the full-length protein (FIG. 4). E. coli expressing the RTX-domain together with GtxC showed haemolytic activity on blood-agar plates and in liquid haemolysis assays, thus, the RTX-domain is a functional haemolytic protein by itself and the N-terminal domain is not essential for the lysis of red blood cells. However, the RTX-domain did exhibit a markedly lower haemolytic activity than the whole toxin, indicating that the N-terminal domain is required for the full haemolytic activity. No cytotoxic activity was detected from interactions between the RTX-domain and HD11 cells suggesting that the N-terminal domain plays an essential role for leukotoxicity. Immunoblotting (FIG. 5) showed that the RTX-domain was expressed and exported; therefore the differences in activity were not due to major differences in expression levels. E. coli expressing only the N-terminal domain had no haemolytic or cytotoxic activity (FIG. 4). However, SDS-PAGE showed that this recombinant protein was primarily present in whole cell fraction and only in diminutive amounts in the extracellular protein fraction (data not shown). We therefore tested the activity of lysates (generated by FastPrep bead beating or lysozyme/sonication treatment) of cells expressing the N-terminal domain, and these did not show any activity of the in liquid haemolysis assays or towards avian macrophages, despite prolonged incubation. These results indicate that the N-terminal domain has no cytolytic activity by itself.

### Example 2

### Detection of gtxA in Gallibacterium

### Materials and Methods

Chromosomal DNA was purified from Gallibacterium strains with the DNeasy Kit (Invitrogen). Standard PCR was done with chromosomal DNA as template (50 ng/50 μl reaction) and the primers rtxA3368F 5′-CAAACCTAATTCAATCGGATG-3′ (SEQ ID NO: 24) and rtxA4625R 5′-TGCTTCAATAATTTTCCATTTTC-3′ (SEQ ID NO: 25) amplifying nucleotides 3332 to 4589 of gtxA. The PCR conditions where; 4 min at 94° C., 35 cycles of 30 sec at 94° C., 30 sec at 51° C. and 105 sec at 72° C. Products were visualised by gel electrophoresis and ethidium bromide staining. The PCR products were sequenced by BigDye cycle sequencing (Macrogen, Korea). Nucleotide sequences where translated and aligned with the CLC workbench.

### Results

Alignment of GtxA toxins from different Gallibacterium strains is presented in FIG. 6.

### Example 3

### Sequences

The sequences listed below refer to sequences of amino acids and nucleic acids mentioned in the application.

Full length GtxA from Gallibacterium anatis, strain 12656-12
(amino acids 1-2026)
>GtxA aa1-2026
SEQ ID No. 1
MLSLKEKVTGIDFDAIKDKVVSLKNTVSNIDFNLVKEDISSLKSNALSIAASDFKNKPVLFKDS
LDLLTDATNTLRKITNQMSSISEISNKSLDLLDSLFEAAKDIVNIAYSKGGVEITKSATELAAK
AALIVDKSIILANKDNTISEAVYHSINNSLQNIQKTAINIATHSHNEDKAEIAKASFELLSQVS
DVISNALKNSGDIGIESQLLADINQFSHSILNTAKTVTDIATMDMNDKTSIAKNSISLIANVND
VISDILVMTDKDTELLNAIHNVTAKNLQNIEESAVNLANADVLSQEGKVSIAINSLTLISQTNK
IVAQVLNEANLSTDKTQFVGELTDVLLNTAKSITLLATGNNATTAGKEQLAVASTNLIGNVNDL
IQSITSFKGKEDIGNALHSAVDGQLSQIKQLAVALSNSNLDSSQGKTAIAITSFGLIAQANNII
NKFLDNMSLSTNVSKSVHSLTNSALDAAKILTNVVQVDANNNQGKVVIANSSLELSKTASDIVS
TVLKSTSISTQHIDIIHNAVNKTLTEMKDSAVAIALASSENNSAEIATHSLSLLSDASNMLKDI
MQGMSPNNVIAPKTLELFNSLFATAQNIVQLADAKSSENIAKASVDLVQSATIILNNVLTLANV
DSSLSKAFHQSFDASVSQIKEVAAQLATASSASNKAEIAKLSFDFISQVSDLATNTLTTAKTGL
DSTLLNNVNGLSHSVLNAAKSVTDIIVSDNPANTASLSVSLVNNANEIVSNILTLSGKQNTLST
AVHDVTAKHLAPIEKIAINLANADNSSSDGKVAIALNSLTLIAQSNHLIEEVLKEAKLDNAKSA
FAHNLTDLVLDTAKTITALASADTSKVDGKQQIASASTHLVGQINEIVKSITTITNSETKVGNA
AYQALKTHLEQVETIAVKLAAANASTAEGRTEIAIESFNLIAKTNGIMTDFLNQIGIKEELTKP
IQGLSNSILDTAKTLTYVVQIDPTTDKGKLSIADSSFELAKSANQIVSYIMDLSGSSSELSHNI
ANTAHQILSISQDRLLSIGNNISALANADKLTKEGVKIIVDSSFAITSDVNGFITDVVKTVGKD
GNPKVGSALSLSNSIIDMGHSIANLIQSDVNTSSGKAAIAEGSIKLIGNINGLVSDVLSLSNAS
TAVSEAISSSAGGILTNLSSLIGSSIKLHNWSNMTQADQIAVGFDIGLKAVSTIATGVGTTAQS
IAKIIGITTMLPQIGAAVSGIALAASPLEIKGLVDEHKYVKQIDSLASETKTYGYQGDELLASL
LNEKFALNTAYTATDIALNLATTAISVAATASVIGAPIAAIAGVVRGAIGGIMSAIKQPALEHI
AKRYVDQIEKYGDIQKYFDQNTEATLNKFYASQEVIQSFKQLQKLYNVDNIITLDGVASSDSAI
ELAAITKLVEQMNKANNYAQLIRNGEIDKALSAQYLSMDAKTGVLEITAPGNSLIKFNSPLFAP
GVEEARRKAVGKNNFYTDLIINGPNEHTINDGAGNNIFISNDKYASVLYDENGKLLKHINLNIN
AGDGNDTYIADNGHSLFNGGNGTDSVSYNNEHIHGIVVHGRDAGTYSVTKHIADAEVTVENIKV
KNHQYGKRQERVEYRELHIETKSYDASDMLYNVEVISASDYDDVMYGSKGNDYFLAQNGNDLVY
GKEGDDIIFGGAGDDKLYGETGNDTLNGGLGKDLIYGGEGNDTIIQDDALSSDTIFGDEGIDTL
DLRHLVINDEGLGVVADLQSEKLYKGTIFDHIYDIENIIGTSGNDNLIGNHKDNILIGNDGDDI
LEGYSGNDVLAGGSGINKLYGGQGADIYLLSTNATNYIFDLTKNNLAKLENSEDLNLQFTKDSD
DNVTLSFNKDGNTIGKTIIEKSSQFGTFSIGDGYYLDLNDGKFKYILSGESSNADLAQNTLHFN
KGEELQVHAAAKDNQIILDHEHQHYINIYSNTQTNIKGFEVGKDKLQLSLLSNNLSSDTKLKFS
GYDIEGGDVNITSGNTYITLSGAGHTDYASKTFNELVTMYLV
C-terminal part of GtxA from Gallibacterium anatis, strain 12656-12
(amino acids 950-2026)
>GtxA aa950-2026
SEQ ID No. 2
QIGIKEELTKPIQGLSNSILDTAKTLTYVVQIDPTTDKGKLSIADSSFELAKSANQIVSYIMDL
SGSSSELSHNIANTAHQILSISQDRLLSIGNNISALANADKLTKEGVKIIVDSSFAITSDVNGF
ITDVVKTVGKDGNPKVGSALSLSNSIIDMGHSIANLIQSDVNTSSGKAAIAEGSIKLIGNINGL
VSDVLSLSNASTAVSEAISSSAGGILTNLSSLIGSSIKLHNWSNMTQADQIAVGFDIGLKAVST
IATGVGTTAQSIAKIIGITTMLPQIGAAVSGIALAASPLEIKGLVDEHKYVKQIDSLASETKTY
GYQGDELLASLLNEKFALNTAYTATDIALNLATTAISVAATASVIGAPIAAIAGVVRGAIGGIM
SAIKQPALEHIAKRYVDQIEKYGDIQKYFDQNTEATLNKFYASQEVIQSFKQLQKLYNVDNIIT
LDGVASSDSAIELAAITKLVEQMNKANNYAQLIRNGEIDKALSAQYLSMDAKTGVLEITAPGNS
LIKFNSPLFAPGVEEARRKAVGKNNFYTDLIINGPNEHTINDGAGNNIFISNDKYASVLYDENG
KLLKHINLNINAGDGNDTYIADNGHSLFNGGNGTDSVSYNNEHIHGIVVHGRDAGTYSVTKHIA
DAEVTVENIKVKNHQYGKRQERVEYRELHIETKSYDASDMLYNVEVISASDYDDVMYGSKGNDY
FLAQNGNDLVYGKEGDDIIFGGAGDDKLYGETGNDTLNGGLGKDLIYGGEGNDTIIQDDALSSD
TIFGDEGIDTLDLRHLVINDEGLGVVADLQSEKLYKGTIFDHIYDIENIIGTSGNDNLIGNHKD
NILIGNDGDDILEGYSGNDVLAGGSGINKLYGGQGADIYLLSTNATNYIFDLTKNNLAKLENSE
DLNLQFTKDSDDNVTLSFNKDGNTIGKTIIEKSSQFGTFSIGDGYYLDLNDGKFKYILSGESSN
ADLAQNTLHFNKGEELQVHAAAKDNQIILDHEHQHYINIYSNTQTNIKGFEVGKDKLQLSLLSN
NLSSDTKLKFSGYDIEGGDVNITSGNTYITLSGAGHTDYASKTFNELVTMYLV
N-terminal part of GtxA from Gallibacterium anatis, strain 12656-12
(amino acids 1-949)
>GtxA aa1-949
SEQ ID No. 3
MLSLKEKVTGIDFDAIKDKVVSLKNTVSNIDFNLVKEDISSLKSNALSIAASDFKNKPVLFKDS
LDLLTDATNTLRKITNQMSSISEISNKSLDLLDSLFEAAKDIVNIAYSKGGVEITKSATELAAK
AALIVDKSIILANKDNTISEAVYHSINNSLQNIQKTAINIATHSHNEDKAEIAKASFELLSQVS
DVISNALKNSGDIGIESQLLADINQFSHSILNTAKTVTDIATMDMNDKTSIAKNSISLIANVND
VISDILVMTDKDTELLNAIHNVTAKNLQNIEESAVNLANADVLSQEGKVSIAINSLTLISQTNK
IVAQVLNEANLSTDKTQFVGELTDVLLNTAKSITLLATGNNATTAGKEQLAVASTNLIGNVNDL
IQSITSFKGKEDIGNALHSAVDGQLSQIKQLAVALSNSNLDSSQGKTAIAITSFGLIAQANNII
NKFLDNMSLSTNVSKSVHSLTNSALDAAKILTNVVQVDANNNQGKVVIANSSLELSKTASDIVS
TVLKSTSISTQHIDIIHNAVNKTLTEMKDSAVAIALASSENNSAEIATHSLSLLSDASNMLKDI
MQGMSPNNVIAPKTLELFNSLFATAQNIVQLADAKSSENIAKASVDLVQSATIILNNVLTLANV
DSSLSKAFHQSFDASVSQIKEVAAQLATASSASNKAEIAKLSFDFISQVSDLATNTLTTAKTGL
DSTLLNNVNGLSHSVLNAAKSVTDIIVSDNPANTASLSVSLVNNANEIVSNILTLSGKQNTLST
AVHDVTAKHLAPIEKIAINLANADNSSSDGKVAIALNSLTLIAQSNHLIEEVLKEAKLDNAKSA
FAHNLTDLVLDTAKTITALASADTSKVDGKQQIASASTHLVGQINEIVKSITTITNSETKVGNA
AYQALKTHLEQVETIAVKLAAANASTAEGRTEIAIESFNLIAKTNGIMTDFLN
Full length gtxA from Gallibacterium anatis, strain 12656-12
(nucleotides 1-6078)
>gtxA nt1-6078
SEQ ID No. 4
GTGCTTTCATTAAAAGAAAAAGTAACTGGAATAGATTTTGATGCAATCAAAGATAAAGTCGTTT
CATTAAAAAACACGGTTTCAAATATTGATTTTAATCTGGTTAAAGAAGATATTTCTTCTTTAAA
AAGCAATGCGTTATCCATCGCGGCATCAGATTTTAAAAATAAACCGGTGTTATTCAAAGACTCT
TTAGACTTACTTACTGATGCTACAAATACACTCAGAAAGATTACCAATCAAATGTCATCAATTA
GCGAAATTTCTAATAAGTCATTAGATTTGCTGGATTCTCTTTTTGAGGCTGCCAAAGATATTGT
AAACATTGCCTATTCAAAAGGTGGTGTCGAAATTACTAAGTCTGCGACAGAATTAGCGGCAAAA
GCGGCATTAATTGTTGATAAAAGTATCATATTAGCAAATAAAGATAATACAATTAGTGAAGCTG
TTTATCATTCTATTAACAACTCATTACAAAATATTCAAAAAACAGCTATCAATATTGCTACACA
TTCACATAATGAAGATAAAGCTGAAATTGCTAAAGCCTCTTTTGAGCTGTTATCTCAAGTTAGT
GATGTTATCAGTAATGCGTTAAAAAATTCAGGTGATATAGGTATCGAATCACAACTCTTAGCCG
ATATTAATCAGTTTTCTCATTCTATTTTGAACACAGCTAAAACAGTTACTGATATAGCTACTAT
GGATATGAATGATAAAACCTCAATCGCTAAAAATAGCATTTCATTAATAGCCAATGTGAATGAT
GTTATTTCCGATATTCTAGTAATGACGGATAAAGACACCGAATTATTAAATGCAATTCATAATG
TTACTGCGAAAAATCTACAGAATATCGAAGAGAGTGCGGTCAATCTTGCAAATGCTGATGTGCT
GTCTCAAGAAGGCAAAGTCAGTATTGCCATTAATTCTTTAACTTTAATATCACAAACCAACAAA
ATTGTTGCGCAAGTGCTAAATGAAGCTAATTTAAGCACTGATAAAACCCAATTTGTTGGCGAAT
TAACCGATGTATTATTGAATACCGCAAAAAGTATTACATTGTTAGCTACCGGTAATAATGCGAC
AACAGCAGGAAAGGAACAGCTGGCAGTTGCCTCAACCAATCTTATTGGTAACGTGAATGATCTC
ATTCAATCAATTACCAGCTTTAAAGGCAAAGAAGATATTGGTAACGCTTTACACAGTGCGGTGG
ACGGACAATTATCACAAATCAAACAACTTGCGGTCGCGTTATCAAACAGTAATCTTGATTCTTC
aCAAGGTAAAACTGCAATAGCCATCACCTCTTTCGGCTTGATTGCACAAGCAAATAATATTATC
AATAAATTCTTGGATAATATGAGTTTAAGTACTAATGTGAGTAAATCGGTTCATAGTTTGACTA
ATTCAGCGCTAGATGCAGCCAAAATTCTCACAAACGTAGTACAAGTAGATGCTAATAACAATCA
AGGAAAGGTCGTGATTGCCAATAGTTCATTAGAACTTTCTAAAACAGCAAGTGATATTGTGTCT
ACTGTGTTAAAAAGCACATCTATTTCAACACAACATATTGATATAATTCATAATGCAGTAAATA
AAACATTAACAGAAATGAAAGATAGTGCGGTAGCAATAGCACTTGCTTCATCTGAAAATAATAG
CGCTGAAATTGCAACGCATTCATTAAGTCTGTTATCCGATGCAAGTAATATGTTGAAAGATATT
ATGCAAGGAATGAGCCCTAATAATGTCATTGCTCCGAAAACATTAGAATTATTTAACTCACTAT
TTGCGACAGCTCAAAATATCGTTCAATTAGCTGACGCAAAATCTTCAGAAAACATTGCTAAAGC
TAGTGTTGATTTGGTACAAAGCGCAACGATTATCCTCAATAACGTATTAACGTTGGCTAACGTT
GATTCTTCTTTAAGTAAAGCTTTTCATCAATCATTTGATGCTTCAGTTTCTCAAATTAAAGAGG
TAGCAGCTCAATTAGCTACCGCGTCTTCTGCCTCTAATAAAGCTGAGATTGCAAAACTCTCTTT
TGATTTTATTAGTCAAGTAAGTGATTTAGCGACCAACACCTTAACAACAGCGAAAACCGGATTA
GATAGCACGCTGCTGAATAACGTTAACGGTCTTTCTCATTCCGTCTTAAATGCAGCAAAATCAG
TAACCGATATTATTGTGAGTGATAACCCAGCGAATACCGCCAGTTTATCCGTTTCTTTGGTGAA
TAATGCCAATGAAATTGTTTCAAATATCTTAACCTTATCCGGAAAACAAAATACGCTCTCCACG
GCAGTACACGATGTAACCGCTAAACATTTAGCGCCGATTGAGAAAATAGCAATTAACCTTGCGA
ATGCCGATAACTCAAGCAGTGATGGAAAAGTTGCTATTGCGTTAAACTCATTAACATTGATTGC
ACAAAGTAACCATTTAATCGAAGAAGTATTAAAAGAGGCTAAATTAGATAATGCGAAGAGCGCC
TTTGCTCACAATTTAACGGATTTAGTATTAGATACCGCCAAAACAATCACGGCATTAGCATCAG
CGGATACCAGTAAAGTAGACGGCAAGCAGCAGATTGCCTCTGCATCAACACATTTAGTCGGACA
AATTAATGAGATTGTCAAATCAATCACGACAATAACCAATTCAGAAACGAAAGTCGGCAATGCC
GCATATCAAGCGTTAAAAACACATTTAGAGCAGGTAGAAACAATTGCGGTTAAACTTGCCGCCG
CCAATGCATCAACAGCGGAAGGCAGAACAGAAATTGCGATTGAATCTTTCAATTTAATCGCAAA
AACCAATGGCATAATGACCGATTTCCTAAATCAAATCGGCATAAAAGAAGAGTTAACCAAACCA
ATCCAAGGTTTATCTAATTCTATTTTAGATACTGCCAAAACCTTAACTTATGTTGTACAAATTG
ACCCAACAACAGACAAAGGTAAACTTTCTATCGCAGATAGCTCATTTGAATTGGCAAAATCTGC
TAACCAAATTGTCTCATATATTATGGATTTATCCGGCAGCTCAAGTGAACTAAGCCATAATATT
GCGAATACGGCTCATCAAATCTTGTCTATATCCCAAGACAGACTATTAAGCATTGGAAATAATA
TTTCTGCATTGGCAAATGCCGATAAACTCACAAAAGAAGGCGTGAAAATTATTGTAGACAGTTC
ATTTGCCATCACCAGCGATGTAAATGGCTTTATCACTGATGTGGTGAAAACAGTAGGCAAAGAC
GGCAATCCTAAAGTGGGGTCAGCCCTATCATTATCCAACTCCATTATTGATATGGGACATTCTA
TCGCAAACCTAATTCAATCGGATGTTAATACAAGTAGCGGGAAAGCGGCTATTGCAGAAGGATC
AATTAAATTAATTGGCAATATTAACGGATTAGTCAGCGATGTGTTAAGCCTTTCTAATGCCTCT
ACCGCCGTTTCTGAAGCTATAAGTTCTTCTGCGGGAGGTATTTTAACTAATTTATCTTCTTTGA
TAGGTTCATCAATTAAACTTCATAATTGGTCTAATATGACCCAAGCCGATCAAATTGCAGTGGG
GTTTGATATTGGATTGAAAGCGGTAAGCACTATTGCGACAGGAGTTGGCACAACAGCACAATCC
ATTGCAAAAATAATTGGTACTACTACGATGTTGCCACAAATTGGTGCTGCTGTATCAGGAATCG
CTCTGGCAGCAAGTCCGTTAGAGATAAAAGGCTTAGTTGACGAACATAAATATGTAAAACAAAT
TGATTCTCTTGCATCAGAAACAAAAACTTATGGTTATCAAGGTGATGAATTATTAGCCAGCCTT
TTAAATGAAAAATTTGCACTTAATACCGCATATACAGCTACAGACATTGCTTTAAATTTAGCAA
CTACAGCGATCTCTGTGGCAGCAACAGCAAGTGTCATTGGTGCACCGATTGCGGCAATTGCAGG
TGTAGTGAGAGGAGCTATCGGCGGTATTATGTCGGCGATCAAACAACCAGCATTGGAACATATT
GCTAAACGTTATGTCGATCAAATTGAAAAGTATGGTGATATTCAAAAATACTTTGATCAAAATA
CTGAGGCAACATTAAATAAATTCTATGCGAGTCAGGAAGTCATTCAATCATTTAAGCAATTACA
GAAATTATATAATGTTGACAACATTATCACCCTTGATGGCGTTGCCAGCTCAGACAGTGCGATA
GAATTAGCCGCTATTACCAAATTAGTTGAGCAAATGAATAAAGCAAATAATTATGCTCAACTTA
TTCGTAACGGTGAAATCGATAAAGCTCTCAGCGCTCAATATTTGAGTATGGATGCTAAAACGGG
CGTGTTGGAAATTACCGCACCAGGCAATTCATTAATAAAATTTAATAGTCCTCTGTTTGCGCCG
GGTGTTGAAGAAGCGCGCAGAAAAGCGGTTGGCAAAAATAATTTTTATACCGATCTTATTATTA
ATGGTCCAAATGAACATACTATTAATGACGGCGCAGGTAATAATATTTTTATTTCTAATGATAA
ATATGCCTCTGTTTTATATGACGAAAATGGaAAATTATTGAAGCATATTAACCTCAATATCAAT
GCCGGCGATGGAAATGATACTTATATTGCTGATAACGGACATTCTCTATTTAATGGAGGTAATG
GAACAGATAGTGTAAGTTATAATAATGAACATATTCACGGCATTGTTGTTCATgGGCGAGATGC
CGGTACTTATTCTGTAACaAAACATATTGCTGATGCAGAAGTAACTGTTGAAAATATTAAAGTG
AAAAATCATCAATACGGTAAACGACAAGAACGAGTTGAGTATAGAGAGTTACATATTGAAACAA
AATCTTATGATGCAAGCGATATGCTTTACAACGTCGAAGTTATCAGTGCCAGTGACTATGATGA
TGTTATGTATGGTAGTAAAGGTAATGATTACTTCCTCGCACAAAATGGTAATGATCTTGTTTAT
GGTAAAGAGGGTGATGACATTATTTTCGGTGGTGCTGGTGATGATAAACTTTATGGAGAAACCG
GTAACGACACCTTAAATGGCGGTTTAGGCAAAGATTTAAtTTATGGTGGCGAAGGAAATGATAC
CATTATTCAAGATGATGCTCTTAGTTCCGACACTATCTTCGGCGATGAAGGGATTGATACATTA
GATCTTCGTCATTTAGTGATTAATGATGAAGGACTCGGTGTTGTTGCTGATCTCCAGTCCGAGA
AGCTTTATAAAGGAACCATCTTTGATCATATCTATGATATAGAGAATATTATAGGTACATCAGG
AAATGATAACCTTATTGGAAATCATAAAGATAATATCTTAATCGGTAATGATGGTGATGATATT
TTAGAAGGCTATAGCGGTAATGATGTACTTGCCGGAGGAAGTGGCATAAATAAACTTTATGGCG
GACAGGGAGCAGATATTTATCTCTTATCCACCAACGCCACAAATTATATCTTCGATCTGACAAA
AAATAATTTAGCAAAATTAGAGAATTCAGAAGATCTTAACCTTCAATTCACTAAAGATAGCGAT
GACAATGTTACCTTATCTTTCAATAAAGATGGCAATACTATCGGTAAAACCATTATAGAAAAAT
CGAGCCAATTTGGCACATTCTCAATAGGTGATGGATATTACTTAGATCTTAATGATGGCAAATT
TAAGTATATTTTATCCGGAGAAAGCTCCAATGCCGATTTAGCTCAAAATACATTACAtTTTAAT
AAAgGTGAAGAACTTCAAGTTCATGCTGCTGCCAAGGATAACCAAATTATATTAGATCACGAAC
ATCAGCATTACATTAATATTTATAGTAATACACAGACTAATATTAAAGGCTTTGAAGTTGGTAA
AGATAAGCTGCAATTATCACTGCTCAGTAATAATTTAAGCTCAGACACCAAATTAAAATTCAGC
GGTTATGATATTGAAGGAGGCGATGTTAATATTACTTCCGGCAATACCTATATTACGTTGTCCG
GTGCGGGGCATACAGATTATGCGAGCAAAACATTTAATGAACTGGTCACTATGTATCTTGTT
Truncated gtxA from Gallibacterium anatis, strain 12656-12
(nucleotides 2848-6078)
>gtxA nt2848-6078
SEQ ID No. 5
CAAATCGGCATAAAAGAAGAGTTAACCAAACCAATCCAAGGTTTATCTAATTCTATTTTAGATA
CTGCCAAAACCTTAACTTATGTTGTACAAATTGACCCAACAACAGACAAAGGTAAACTTTCTAT
CGCAGATAGCTCATTTGAATTGGCAAAATCTGCTAACCAAATTGTCTCATATATTATGGATTTA
TCCGGCAGCTCAAGTGAACTAAGCCATAATATTGCGAATACGGCTCATCAAATCTTGTCTATAT
CCCAAGACAGACTATTAAGCATTGGAAATAATATTTCTGCATTGGCAAATGCCGATAAACTCAC
AAAAGAAGGCGTGAAAATTATTGTAGACAGTTCATTTGCCATCACCAGCGATGTAAATGGCTTT
ATCACTGATGTGGTGAAAACAGTAGGCAAAGACGGCAATCCTAAAGTGGGGTCAGCCCTATCAT
TATCCAACTCCATTATTGATATGGGACATTCTATCGCAAACCTAATTCAATCGGATGTTAATAC
AAGTAGCGGGAAAGCGGCTATTGCAGAAGGATCAATTAAATTAATTGGCAATATTAACGGATTA
GTCAGCGATGTGTTAAGCCTTTCTAATGCCTCTACCGCCGTTTCTGAAGCTATAAGTTCTTCTG
CGGGAGGTATTTTAACTAATTTATCTTCTTTGATAGGTTCATCAATTAAACTTCATAATTGGTC
TAATATGACCCAAGCCGATCAAATTGCAGTGGGGTTTGATATTGGATTGAAAGCGGTAAGCACT
ATTGCGACAGGAGTTGGCACAACAGCACAATCCATTGCAAAAATAATTGGTACTACTACGATGT
TGCCACAAATTGGTGCTGCTGTATCAGGAATCGCTCTGGCAGCAAGTCCGTTAGAGATAAAAGG
CTTAGTTGACGAACATAAATATGTAAAACAAATTGATTCTCTTGCATCAGAAACAAAAACTTAT
GGTTATCAAGGTGATGAATTATTAGCCAGCCTTTTAAATGAAAAATTTGCACTTAATACCGCAT
ATACAGCTACAGACATTGCTTTAAATTTAGCAACTACAGCGATCTCTGTGGCAGCAACAGCAAG
TGTCATTGGTGCACCGATTGCGGCAATTGCAGGTGTAGTGAGAGGAGCTATCGGCGGTATTATG
TCGGCGATCAAACAACCAGCATTGGAACATATTGCTAAACGTTATGTCGATCAAATTGAAAAGT
ATGGTGATATTCAAAAATACTTTGATCAAAATACTGAGGCAACATTAAATAAATTCTATGCGAG
TCAGGAAGTCATTCAATCATTTAAGCAATTACAGAAATTATATAATGTTGACAACATTATCACC
CTTGATGGCGTTGCCAGCTCAGACAGTGCGATAGAATTAGCCGCTATTACCAAATTAGTTGAGC
AAATGAATAAAGCAAATAATTATGCTCAACTTATTCGTAACGGTGAAATCGATAAAGCTCTCAG
CGCTCAATATTTGAGTATGGATGCTAAAACGGGCGTGTTGGAAATTACCGCACCAGGCAATTCA
TTAATAAAATTTAATAGTCCTCTGTTTGCGCCGGGTGTTGAAGAAGCGCGCAGAAAAGCGGTTG
GCAAAAATAATTTTTATACCGATCTTATTATTAATGGTCCAAATGAACATACTATTAATGACGG
CGCAGGTAATAATATTTTTATTTCTAATGATAAATATGCCTCTGTTTTATATGACGAAAATGGa
AAATTATTGAAGCATATTAACCTCAATATCAATGCCGGCGATGGAAATGATACTTATATTGCTG
ATAACGGACATTCTCTATTTAATGGAGGTAATGGAACAGATAGTGTAAGTTATAATAATGAACA
TATTCACGGCATTGTTGTTCATgGGCGAGATGCCGGTACTTATTCTGTAACaAAACATATTGCT
GATGCAGAAGTAACTGTTGAAAATATTAAAGTGAAAAATCATCAATACGGTAAACGACAAGAAC
GAGTTGAGTATAGAGAGTTACATATTGAAACAAAATCTTATGATGCAAGCGATATGCTTTACAA
CGTCGAAGTTATCAGTGCCAGTGACTATGATGATGTTATGTATGGTAGTAAAGGTAATGATTAC
TTCCTCGCACAAAATGGTAATGATCTTGTTTATGGTAAAGAGGGTGATGACATTATTTTCGGTG
GTGCTGGTGATGATAAACTTTATGGAGAAACCGGTAACGACACCTTAAATGGCGGTTTAGGCAA
AGATTTAAtTTATGGTGGCGAAGGAAATGATACCATTATTCAAGATGATGCTCTTAGTTCCGAC
ACTATCTTCGGCGATGAAGGGATTGATACATTAGATCTTCGTCATTTAGTGATTAATGATGAAG
GACTCGGTGTTGTTGCTGATCTCCAGTCCGAGAAGCTTTATAAAGGAACCATCTTTGATCATAT
CTATGATATAGAGAATATTATAGGTACATCAGGAAATGATAACCTTATTGGAAATCATAAAGAT
AATATCTTAATCGGTAATGATGGTGATGATATTTTAGAAGGCTATAGCGGTAATGATGTACTTG
CCGGAGGAAGTGGCATAAATAAACTTTATGGCGGACAGGGAGCAGATATTTATCTCTTATCCAC
CAACGCCACAAATTATATCTTCGATCTGACAAAAAATAATTTAGCAAAATTAGAGAATTCAGAA
GATCTTAACCTTCAATTCACTAAAGATAGCGATGACAATGTTACCTTATCTTTCAATAAAGATG
GCAATACTATCGGTAAAACCATTATAGAAAAATCGAGCCAATTTGGCACATTCTCAATAGGTGA
TGGATATTACTTAGATCTTAATGATGGCAAATTTAAGTATATTTTATCCGGAGAAAGCTCCAAT
GCCGATTTAGCTCAAAATACATTACAtTTTAATAAAgGTGAAGAACTTCAAGTTCATGCTGCTG
CCAAGGATAACCAAATTATATTAGATCACGAACATCAGCATTACATTAATATTTATAGTAATAC
ACAGACTAATATTAAAGGCTTTGAAGTTGGTAAAGATAAGCTGCAATTATCACTGCTCAGTAAT
AATTTAAGCTCAGACACCAAATTAAAATTCAGCGGTTATGATATTGAAGGAGGCGATGTTAATA
TTACTTCCGGCAATACCTATATTACGTTGTCCGGTGCGGGGCATACAGATTATGCGAGCAAAAC
ATTTAATGAACTGGTCACTATGTATCTTGTT
Truncated gtxA from Gallibacterium anatis, strain 12656-12
(nucleotides 1-2847)
>gtxA nt1-2847
SEQ ID No. 6
GTGCTTTCATTAAAAGAAAAAGTAACTGGAATAGATTTTGATGCAATCAAAGATAAAGTCGTTT
CATTAAAAAACACGGTTTCAAATATTGATTTTAATCTGGTTAAAGAAGATATTTCTTCTTTAAA
AAGCAATGCGTTATCCATCGCGGCATCAGATTTTAAAAATAAACCGGTGTTATTCAAAGACTCT
TTAGACTTACTTACTGATGCTACAAATACACTCAGAAAGATTACCAATCAAATGTCATCAATTA
GCGAAATTTCTAATAAGTCATTAGATTTGCTGGATTCTCTTTTTGAGGCTGCCAAAGATATTGT
AAACATTGCCTATTCAAAAGGTGGTGTCGAAATTACTAAGTCTGCGACAGAATTAGCGGCAAAA
GCGGCATTAATTGTTGATAAAAGTATCATATTAGCAAATAAAGATAATACAATTAGTGAAGCTG
TTTATCATTCTATTAACAACTCATTACAAAATATTCAAAAAACAGCTATCAATATTGCTACACA
TTCACATAATGAAGATAAAGCTGAAATTGCTAAAGCCTCTTTTGAGCTGTTATCTCAAGTTAGT
GATGTTATCAGTAATGCGTTAAAAAATTCAGGTGATATAGGTATCGAATCACAACTCTTAGCCG
ATATTAATCAGTTTTCTCATTCTATTTTGAACACAGCTAAAACAGTTACTGATATAGCTACTAT
GGATATGAATGATAAAACCTCAATCGCTAAAAATAGCATTTCATTAATAGCCAATGTGAATGAT
GTTATTTCCGATATTCTAGTAATGACGGATAAAGACACCGAATTATTAAATGCAATTCATAATG
TTACTGCGAAAAATCTACAGAATATCGAAGAGAGTGCGGTCAATCTTGCAAATGCTGATGTGCT
GTCTCAAGAAGGCAAAGTCAGTATTGCCATTAATTCTTTAACTTTAATATCACAAACCAACAAA
ATTGTTGCGCAAGTGCTAAATGAAGCTAATTTAAGCACTGATAAAACCCAATTTGTTGGCGAAT
TAACCGATGTATTATTGAATACCGCAAAAAGTATTACATTGTTAGCTACCGGTAATAATGCGAC
AACAGCAGGAAAGGAACAGCTGGCAGTTGCCTCAACCAATCTTATTGGTAACGTGAATGATCTC
ATTCAATCAATTACCAGCTTTAAAGGCAAAGAAGATATTGGTAACGCTTTACACAGTGCGGTGG
ACGGACAATTATCACAAATCAAACAACTTGCGGTCGCGTTATCAAACAGTAATCTTGATTCTTC
aCAAGGTAAAACTGCAATAGCCATCACCTCTTTCGGCTTGATTGCACAAGCAAATAATATTATC
AATAAATTCTTGGATAATATGAGTTTAAGTACTAATGTGAGTAAATCGGTTCATAGTTTGACTA
ATTCAGCGCTAGATGCAGCCAAAATTCTCACAAACGTAGTACAAGTAGATGCTAATAACAATCA
AGGAAAGGTCGTGATTGCCAATAGTTCATTAGAACTTTCTAAAACAGCAAGTGATATTGTGTCT
ACTGTGTTAAAAAGCACATCTATTTCAACACAACATATTGATATAATTCATAATGCAGTAAATA
AAACATTAACAGAAATGAAAGATAGTGCGGTAGCAATAGCACTTGCTTCATCTGAAAATAATAG
CGCTGAAATTGCAACGCATTCATTAAGTCTGTTATCCGATGCAAGTAATATGTTGAAAGATATT
ATGCAAGGAATGAGCCCTAATAATGTCATTGCTCCGAAAACATTAGAATTATTTAACTCACTAT
TTGCGACAGCTCAAAATATCGTTCAATTAGCTGACGCAAAATCTTCAGAAAACATTGCTAAAGC
TAGTGTTGATTTGGTACAAAGCGCAACGATTATCCTCAATAACGTATTAACGTTGGCTAACGTT
GATTCTTCTTTAAGTAAAGCTTTTCATCAATCATTTGATGCTTCAGTTTCTCAAATTAAAGAGG
TAGCAGCTCAATTAGCTACCGCGTCTTCTGCCTCTAATAAAGCTGAGATTGCAAAACTCTCTTT
TGATTTTATTAGTCAAGTAAGTGATTTAGCGACCAACACCTTAACAACAGCGAAAACCGGATTA
GATAGCACGCTGCTGAATAACGTTAACGGTCTTTCTCATTCCGTCTTAAATGCAGCAAAATCAG
TAACCGATATTATTGTGAGTGATAACCCAGCGAATACCGCCAGTTTATCCGTTTCTTTGGTGAA
TAATGCCAATGAAATTGTTTCAAATATCTTAACCTTATCCGGAAAACAAAATACGCTCTCCACG
GCAGTACACGATGTAACCGCTAAACATTTAGCGCCGATTGAGAAAATAGCAATTAACCTTGCGA
ATGCCGATAACTCAAGCAGTGATGGAAAAGTTGCTATTGCGTTAAACTCATTAACATTGATTGC
ACAAAGTAACCATTTAATCGAAGAAGTATTAAAAGAGGCTAAATTAGATAATGCGAAGAGCGCC
TTTGCTCACAATTTAACGGATTTAGTATTAGATACCGCCAAAACAATCACGGCATTAGCATCAG
CGGATACCAGTAAAGTAGACGGCAAGCAGCAGATTGCCTCTGCATCAACACATTTAGTCGGACA
AATTAATGAGATTGTCAAATCAATCACGACAATAACCAATTCAGAAACGAAAGTCGGCAATGCC
GCATATCAAGCGTTAAAAACACATTTAGAGCAGGTAGAAACAATTGCGGTTAAACTTGCCGCCG
CCAATGCATCAACAGCGGAAGGCAGAACAGAAATTGCGATTGAATCTTTCAATTTAATCGCAAA
AACCAATGGCATAATGACCGATTTCCTAAAT

### Example 4

### gtxA Sequence and Function is Conserved Amonghaemolytic Gallibacterium

Sequence Conservation of gtxA Nucleotides 1-950 Between Gallibacterium Strains

BlastN with gtxA nt 1-950 from strain 12656-12 against the draft genome sequences of other Gallibacterium strains.

| Strain | sequence identity |
|---|---|
| 4895 | 316/318 (99%) |
| 07990 | 918/947 (96%) |
| Avicor | 936/950 (98%) |
| 201 | 906/947 (95%) |
| 203 | 615/627 (98%) |
| 204 | 915/947 (96%) |
| 211 | 941/947 (99%) |
| 213 | 621/635 (97%) |

### GtxA is Expressed and Secreted by Haemolytic Gallibacterium

### Materials and Methods

When culturing cells for the isolation of protein, an overnight culture (16 hrs) was diluted approx. 1:100, OD₆₀₀ was adjusted to 0.03, and the bacteria were incubated under moderate aeration (120 rpm) in 30 mL BHI-broth in a 100 mL Erlenmeyer flask. Cells and supernatant was harvested in late exponential phase at OD₆₀₀=0.6(±0.1). Extracellular proteins were prepared from filter-sterilized culture supernatant (low protein binding filter (0.22 μm) (Millex® GP (Millipore)). Proteins were precipitated overnight (−20° C.) by the addition of one volume ice-cold 96% ethanol, collected by centrifugation (13000 g for 30 min. at 0° C.), and resuspended in 10 mM Tris (1/100 of the original volume). Proteins (13 μl) were separated by SDS-PAGE in NuPAGE Novex 3-8% Tris-Acetate Mini Gels in Tris-Acetate SDS Running Buffer (Invitrogen). For Western blotting, proteins were blotted onto a polyvinylidene difluoride (PVDF) membrane (Invitrogen). ApxI antiserum [24] was used as primary antibody, and bound ApxI-antibodies were detected with Western Breeze Chemiluminescent kit (Anti-Rabbit) (Invitrogen). The chemiluminescence signal was captured with the Geliance 600 imaging system (Perkin Elmer Elmer).

GtxA is the Main Cytolytic Toxin in G. anatis

gtxA of eight genetically diverse G. anatis biovar haemolytica strains (10672-6, 10T4, 21K2, 24T10, 4895, 07990, and Avicor) was inactivated by mutation as previously described (Kristensen et al., 2010). All mutants proved nonhaemolytic (data not shown), demonstrating that GtxA is responsible for the haemolytic activity in these strains. This result supports the role of GtxA as the dominant cytolytic toxin in G. anatis.

gtxA is Responsible for Haemolytic Activity in G. genomospecies 1 and G. genomospecies 2.

The presence of gtxA in G. Genomospecies 1 and G. Genomospecies 2 suggested that gtxA was also responsible for the haemolytic activity in G. genomospecies 1 and 2. We constructed gtxA mutants in the G. genomospecies 1 and 2 type strains (CCM5974 and CCM5976) and they were nonhaemolytic (data not shown), demonstrating that GtxA is also responsible for the haemolytic activity in these species.

### Example 5

### Identification and Characterisation of a Type I Secretion Locus (gtxEBD) Required for GtxA Export GtxA is an extracellular protein, but unlike most RTX-toxin in the Pasteurellaceae it is not co-transcribed with components of its cognate type 1 secretion system (T1SS) (FIG. 9). T1SS are composed of multimers of three proteins, an inner membrane ATPase, an inner membrane channel protein and an outer membrane protein, and in E. coli the T1SS proteins secreting the prototypic RTX-toxin a-haemolysin are designated HIyB, HIyD and ToIC, respectively (Holland et al., Mol Membr Biol 2005, 22: 29-39). To identify a TISS responsible for GtxA-export, we searched the genome sequence of strain 12656-12, with the amino acid sequences of E. coli HIyB and HIyD as queries. By this approach, we identified a predicted T1SS-operon without an obvious substrate, and considered it a likely candidate for GtxA secretion. This operon contained three genes, which we named gtxE, gtxB, and gtxD (FIG. 9). The encoded proteins GtxE, GtxB and GtxC were predicted to be an outer membrane protein, an inner membrane ATPase, and an inner membrane protein, respectively, which corresponds to the three components required to constitute a type 1 secretion system. To test whether the operon was needed for GtxA export, we constructed a mutant (ΔgtxBD) by deleting most of gtxB (from nucleotide position 92) and the beginning of gtxD (the first 123 nt) in G. anatis 12656-12.

Construction of ΔgtxBD

For the construction of ΔgtxBD, two PCR-fragments were generated, one (951 bp) with primers 2870E-XbaI (5′-CTGATCTAGACGCCGTAAATCGCATAATCA-3′) (SEQ ID NO: 26) and 3821 R-EcoRI (5′-CGAATTCCCGGCAGAAAAGGTCAACA-3′) (SEQ ID NO: 27) and the other (1233 nt) with 6044-SOE (TTTCTGCCGGGAATTCGGCGAATGGTGTGAGAAG-5′) (SEQ ID NO: 28) and 7267R-SalI (TCAAGTCGACAAGCCAAAGCCAATACGA-3′) (SEQ ID NO: 29). Restriction sites in the primer sequences are underlined. The two PCR-fragments were connected by the Splicing by overlap extension PCR with primers 2870f-XbaI and 7267R-SalI. The resulting 2184 nt-fragment was digested with SalI and XbaI, phosphatase-treated with Antarctic phosphatase (Fermentas), gel-purified and ligated into XbaI/SalI-double digested and gel-purified pBluescript. The kanamycin-cassette (Tn903) from EcoRI-digested pUC4-KISS was gel-purified and ligated into the EcoRI site in the SOEing-fragment. The kanamycin resistance gene was inserted in the same transcriptional direction as gtxB. The plasmid DNA was linearised by digestion with SalI and XbaI and column purified (GFX, Amersham). The natural competence of G. anatis 12656-12 was induced by the MIV-method as previously described for Haemophilus influenzae (Poje & Redfield, 2003); Briefly, G. anatis 12656-12 was grown in BHI to an OD₆₀₀ of 0.2, washed once in MIV and incubated in MIV for 100 minutes. The linear DNA was added to the cells at a concentration of 0.5 μg DNA/ml. After 20 minutes of incubation at 37° C., two volumes of BHI were added and the bacteria were incubated for one hour (37° C.) before the transformants were selected on blood agar plates with 4 μg/ml kanamycin. Transformants were re-streaked on plates with 4 μg/mL kanamycin and the deletion was verified by PCR with the primer pair TISS2607F (5′-TTTCCTGTAATGCCTGCT-3′) (SEQ ID NO: 30) and TISS7572R (5′-TTTTGATCGTTCGGGCTT-3′) (SEQ ID NO: 31) and sequencing of the PCR-product.

### Example 6

### GtxA is not Likely to be Part of a Vaccine Based on Whole Cell Antigen

GtxA is not present in detectable levels in cell lysates and does not accumulate intracellularly in the absence of the type I secretion gtxEBD

We identified a type I secretion system locus (gtxEBD) which is required for the secretion of GtxA.

In the secretion mutant ΔgtxBD (constructed as described in Example 5) the haemolytic activity of ΔgtxBD was highly reduced compared to the wild type (se FIG. 8A). Western blotting showed that the deletion of gtxBD resulted in a lack of GtxA in the supernatant (FIG. 8B). These observations support that gtxEBD is responsible for GtxA export. GtxA was not detected in whole cell protein of wild-type or ΔgtxBD, i.e. GtxA does not accumulate inside the cells in the absence of the secretion system. Similar observations have been made with RTX-toxin secretion-deficient mutants in A. actinomycetemcomitans (leukotoxin LtxA) and E. coli (α-haemolysin HIyA). In these species, the lack of intracellular accumulation of toxin is not due to altered transcription, but presumably a rapid degradation of the toxin in the cytoplasm.

### Example 7

### Infection Trial with a Gallibacterium anatis gtxA Mutant Unable of Expressing GtxA

### Introduction

The cytolytic RTX toxin GtxA has been proposed as a key virulence factor for G. anatis during infections in poultry. To substantiate this, we performed an infection trial in egg-laying chicken, with the purpose of characterizing the contribution of GtxA to the lesions observed during an infection with the wild-type (wt) strain and its isogenic ΔgtxA mutant.

### Materials and Methods

A total of 24 birds were included in the study. All birds were infected via the intraperitoneal route attempting a deep deposition of equal amounts of Gallibacterium anatis at the root of the ovary. 16 birds were infected with G. anatis 12656-12 wt, whereas 16 birds were infected with its isogenic but non-haemolytic ΔgtxA mutant, unable of expressing the RTX GtxA (Kristensen et al., 2010).

### Results

Overview of results obtained from the infection trial.

| | Birds | | |
|---|---|---|---|
| | euthanized PI | | |
| Strain | 48 h | 144 h | Macroscopic lesions and bacteriological culture |
| ΔgtxA | 8 | | 2/8: No lesions |
| | | | 4/8: Non-purulent oophoritis/regressive ovary |
| | | | 2/8: Purulent oophoritis |
| | | 8 | 4/8: No lesions |
| | | | 1/8: Purulent oophoritis |
| | | | 1/8: Purulent oophoritis |
| | | | 2/8: Purulent oophoritis and peritonitis |
| WT | 4 | | 4/4: Purulent oophoritis, salpingitis and |
| | | | peritonitis, |
| | | 4 | 3/4: Purulent oophoritis, salpingitis and |
| | | | peritonitis, |
| | | | 1/4: No lesions |

Birds infected with the wt generally developed a disseminated and purulent inflammation involving the reproductive tract and the peritoneum, corresponding to lesions observed from natural infections with G. anatis in the field.

Birds infected with the ΔgtxA mutant generally developed a milder inflammation localized to the ovary. In a few animals (2/8) the localized inflammation was stronger and purulent at 48 h PI, whereas in 2 out of 8 animals purulent oophoritis and peritonitis was observed at 6 days PI.

It can thus be concluded that GtxA contributes substantially in the pathogenesis of G. anatis in chicken.

1. An isolated polypeptide, said polypeptide comprising an amino acid sequence selected from the group consisting of
a) SEQ ID No. 1, 2 or 3;
b) a sequence variant of the amino acid sequence selected from the group consisting of SEQ ID No. 1, 2 and 3, wherein the variant has at least 70% sequence identity to said SEQ ID No.; and
c) a fragment consisting of a least 150 contiguous amino acids of any of a), wherein any amino acid specified in the chosen sequence is changed to a different amino acid, provided that no more than 30 of the amino acids in the sequence are so changed.

2. The polypeptide of claim 1 that is a naturally occurring allelic variant of the sequence selected from the group consisting of SEQ ID No. 1, 2 and 3

3. The polypeptide of claim 1, wherein said polypeptide comprises an amino acid sequence originating from the family Pasteurellaceae, more preferably from the genus Gallibacterium, more preferably selected from the group of Gallibacterium anatis, Gallibacterium genomospecies 1 and Gallibacterium genomospecies 2.

4. The polypeptide of claim 1 that is a variant polypeptide described therein, wherein any amino acid specified in the chosen sequence is changed to provide a conservative substitution.

5. The polypeptide of claim 1, wherein the signal peptide has been replaced by a heterologous signal peptide.

6. The polypeptide of claim 1, having at least 70% sequence identity to SEQ ID No. 1, more preferably at least 75%, more preferably at least 80%, more preferably at least 85%, more preferably at least 90%, more preferably at least 95%, more preferably at least 98%, more preferably a polypeptide having the sequence of SEQ ID No. 1.

7. The polypeptide of claim 1, having at least 70% sequence identity to SEQ ID No. 2, more preferably at least 75%, more preferably at least 80%, more preferably at least 85%, more preferably at least 90%, more preferably at least 95%, more preferably at least 98%, more preferably a polypeptide having the sequence of SEQ ID No. 2.

8. The polypeptide of claim 1, having at least 70% sequence identity to SEQ ID No. 3, more preferably at least 75%, more preferably at least 80%, more preferably at least 85%, more preferably at least 90%, more preferably at least 95%, more preferably at least 98%, more preferably a polypeptide having the sequence of SEQ ID No. 3.

9. The polypeptide of claim 1, or fragments hereof, wherein said fragments comprises at least 150 amino acids, preferably at least 200 amino acids, more preferably at least 300 amino acids, more preferably at least 500 amino acids, more preferably at least 750 amino acids, more preferably at least 1000 amino acids, more preferably 1250 amino acids, more preferably 1500 amino acids, more preferably 1750 amino acids, most preferably 2000 amino acids.

10. The polypeptide of claim 1, wherein said polypeptide has been specifically modified to remove toxic activity.

11. The polypeptide of claim 1, wherein said variant is biologically active.

12. The polypeptide of claim 11, wherein biological activity is toxicity, wherein said toxicity comprises the forming of a pore in the donee, more preferably cytotoxicity, more preferably cytolytic cytotoxicity, yet more preferably haemolytic cytotoxicity.

13. The polypeptide of claim 9, wherein said variants are immunogenic.

14. The polypeptide of claim 9, wherein said fragments contain no more than 30 amino acid substitutions, more preferably no more than 25, more preferably no more than 20, more preferably no more than 15, more preferably no more than 10, more preferably no more than 5, more preferably no amino acid substitutions compared to SEQ ID NO 1.

15. The polypeptide of claim 1, further comprising an affinity tag, such as a polyHis tag, a GST tag, a HA tag, a FLAG tag, a C-myc tag, a HSV tag, a V5 tag, a maltose binding protein tag, a cellulose binding domain tag, a BCCP tag, a Calmodulin tag, a Nus tag, a Glutathione-S-transferase tag, a Green fluorescent protein tag, a Thioredoxin tag, a S tag, a Strep tag.

16. The polypeptide of claim 15, wherein any affinity tag is cleavable.

17. The polypeptide of claim 1 being inactivated.

18. The polypeptide of claim 17, wherein said polypeptide is inactivated by heat or radiation.

19. The polypeptide of claim 17, wherein said polypeptide is chemically inactivated, preferably by exposure to formaldehyde.

20. The polypeptide of claim 17, wherein said polypeptide is a non-acylated form of SEQ ID No. 1

21. The polypeptide of claim 17, wherein said polypeptides or any fragment hereof is immunogenic.

22-96. (canceled)
